(19) United States
(12) Patent Application Publication
(10) Pub. No.: US 2026/0258319 A1
(43) Pub. Date: Sep. 3, 2026

(54) LUBRICANT FOR MAGNETIC RECORDING MEDIA

(71) Applicant: Resonac Corporation, Tokyo (JP)

(72) Inventors: Daisuke YAGYU, Minato-ku, Tokyo (JP); Tsuyoshi KATO, Minato-ku, Tokyo (JP); Takuma KURODA, Minato-ku, Tokyo (JP); Nobuo OSAWA, Minato-ku, Tokyo (JP); Masataka UMETANI, Minato-ku, Tokyo (JP)

(73) Assignee: Resonac Corporation, Tokyo (JP)

(21) Appl. No.: 18/861,630

(22) PCT Filed: Mar. 26, 2024
(86) PCT No.: PCT/JP2024/011888

§ 371 Date: Oct. 30, 2024

(30) Foreign Application Priority Data
Apr. 12, 2023 (JP) 2023-065258

(51) Int. Cl. C10M 147/04; C08G 65/48; C10N 40/18; G11B 5/725; G11B 5/84

(52) U.S. Cl. CPC C10M 147/04; C08G 65/48; G11B 5/7257; G11B 5/8408; C08G 2650/48; C10N 2040/18

(57) ABSTRACT

A lubricant for magnetic recording media containing a fluorine-containing ether compound represented by the following formula (1),

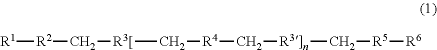

wherein n is any one of integers of 0 to 2. R³ and R^{3′} are each independently a predetermined perfluoropolyether chain; R⁴ is a divalent linking group having 1 to 3 polar groups; R² and R⁵ are divalent linking groups having one or more polar groups, and are the same or different; an end of R² on a side bonded to R¹ is an oxygen atom; an end of R⁵ on a side bonded to R⁶ is an oxygen atom; R¹ and R⁶ are each a terminal group bonded to an oxygen atom at an end of R² or R⁵; and R¹ and R⁶ are hydrogen atoms or organic groups having 1 to 50 carbon atoms.

### TECHNICAL FIELD

The present invention relates to a lubricant for magnetic recording media and a production method thereof, and a magnetic recording medium and a production method thereof.

### BACKGROUND ART

In recent years, development of low-cost, large-capacity magnetic recording media for data centers have been progressing.

A magnetic recording medium has been known, in which a magnetic layer (magnetic recording layer) has been stacked on a substrate, a protective layer such as carbon is then formed on the magnetic layer, and a lubricating layer is further formed on the protective layer. The protective layer protects information recorded on the magnetic layer and improves sliding properties of a magnetic head of a magnetic recording and reproduction apparatus. The lubricating layer prevents direct contact between the magnetic head and the protective layer, significantly reducing frictional force of a sliding magnetic head.

In addition to wear resistance, the lubricating layer arranged on the outermost surface of the magnetic recording medium is required to have various properties such as resistance to chemical substances (prevention of contamination by siloxane and the like), long-term stability, and heat resistance.

The lubricating layer has been coated with a lubricant for magnetic recording media (hereinafter may be abbreviated as “lubricant”). In recent years, a fluorinated polymer containing a perfluoropolyether (hereinafter may be abbreviated as a “PFPE”) chain with a perfluorooxyalkylene group as a constituent repeating unit and having a polar group such as a hydroxy group at an end, has been used as a lubricant.

For production of fluorinated polymer used in the lubricant, a PFPE polymer having a hydroxy group at its end is used as a raw material. Specific examples of the PFPE polymers include Krytox (registered trademark) manufactured by Chemours Inc., Fomblin (registered trademark) manufactured by Solvay Solexis, Inc., and Demnum (registered trademark) manufactured by Daikin Industries, Ltd. Krytox (registered trademark) has a repeating unit of —[CF(CF₃)CF₂O]— and is produced through a ring-opening polymerization step of hexafluoropropylene oxide. Fomblin (registered trademark) is a copolymer in which two types of repeating units of —[CF(Z)CF₂O]— (wherein Z represents a fluorine atom or CF₃) and —[CF₂O]—, are randomly arranged, and is produced through photochemical oxidative polymerization of tetrafluoroethylene or hexafluoropropylene. Demnum (registered trademark) has a repeating unit of —[CF₂CF₂CF₂O]— and is produced via a step of ring-opening polymerization of 2,2,3,3-tetrafluorooxetane. Moreover, as a specific example of another PFPE polymer, a fluorinated polyethylene glycol having a repeating unit of —[CF₂CF₂O]—, is known to be synthesized by liquid-phase fluorination of a polyethylene glycol derivative.

Thus, the PFPE polymer is produced by polymerizing fully or partially fluorinated monomers (since the partially remaining protons are completely replaced with fluorine atoms, a liquid-phase fluorination step may be added after polymerization.), or by liquid-phase fluorinating a non-fluorinated polymer obtained by a polymerization reaction.

Therefore, the PFPE polymer produced by such steps is a mixture of components with different numbers of repetitions and has a wide molecular weight distribution.

A fluorinated polymer for lubricants which has been produced using these PFPE polymers as raw materials is also a mixture of components with different numbers of repetitions and has a wide molecular weight distribution of Mw/Mn=approximately 1.1 to 1.5. As the fluorinated polymer for lubricants, for example, compounds described in Patent Literatures 1 to 3 have been known.

Patent Literature 1 describes a fluorine-containing ether compound having one perfluoropolyether chain. As a raw material thereof, a PFPE polymer that is a mixture of polymers having a repeating unit of —(CF₂CF₂CF₂O)— and the number of repetitions of 1 to 7, and having a molecular weight distribution of 1.2, is described.

Patent Literature 2 describes a fluorine-containing ether compound having two perfluoropolyether chains. Patent Literature 3 describes a fluorine-containing ether compound having three perfluoropolyether chains. As a raw material thereof, a PFPE polymer having a molecular weight distribution of 1.1 is described.

Various properties required for a lubricating layer, such as wear resistance, resistance to chemical substances, and long-term stability are known to vary greatly depending on the molecular weight of a fluorinated polymer used in a lubricant. It is therefore of importance to control the molecular weight distribution of the fluorinated polymer within a desired range.

As a method for coating a surface of a protective layer of a magnetic recording medium with a lubricant, a dipping method is generally used. In the case of using the dipping method, for example, the following method can be employed. First, a lubricant is dissolved in a fluorine-based solvent such as Vertrel (registered trademark) XF (product name, manufactured by Chemours-Mitsui Fluoroproducts Co., Ltd.) to prepare a lubricating layer forming solution so as to have a concentration suitable for coating. Thereafter, a substrate on which each layer up to the protective layer has been formed is immersed in the lubricating layer forming solution placed in an immersion tank of a dip coater. Next, the substrate is pulled up from the immersion tank at a predetermined speed. This enables coating a surface of the protective layer with the lubricant.

### CITATION LIST

### Patent Literature

[Patent Literature 1] JP2018-024614A
[Patent Literature 2] WO2021/020066
[Patent Literature 3] WO2022/215726

### SUMMARY OF INVENTION

### Technical Problem

In a lubricant coating step by the above-mentioned dipping method, the lubricating layer forming solution placed in the immersion tank is repeatedly used for a certain period of time, and several thousand to several hundred thousand substrates undergo processing. However, when a conventional fluorinated polymer having a wide molecular weight distribution is used as a lubricant, there has been a problem that as the number of substrates coated increases, the number-average molecular weight of the fluorinated polymer contained in a lubricating layer increases. This is because in the coating step, fluorinated polymers with small molecular weights have been consumed first and adsorbed onto a protective layer, resulting in a molecular weight distribution of the fluorinated polymer in the lubricating layer forming solution being biased toward the larger molecular weight side. The fluorinated polymer with small molecular weight has a large proportion of polar groups such as hydroxy groups relative to the overall molecule size, and has high polarity as entire molecule. Therefore, the fluorinated polymer with small molecular weight has strong adsorption force to a protective layer and is adsorbed onto the protective layer before a fluorinated polymer with large molecular weight. Therefore, as the number of substrates coated in the coating step increases, a proportion of a fluorinated polymer with small molecular weight in the lubricating layer forming solution decreases significantly.

As the number-average molecular weight of a fluorinated polymer in the lubricating layer increases, its wear resistance and resistance to chemical substances are known to deteriorate. As a result, the lubricating layer forming solution in an immersion tank needs to be frequently exchanged, causing a decrease in productivity in the coating step. Also, only a portion of the fluorinated polymers contained in the lubricating layer forming solution is coated as a lubricating layer, and the remaining components are not used and discarded together with a fluorine-based solvent, making this undesirable from the standpoint of production costs and environmental load.

An object of the present invention is to provide a lubricant for magnetic recording media capable of maintaining wear resistance and resistance to chemical substances of a lubricating layer without deteriorating even though the number of substrates coated increases in a coating step of the lubricant.

### Solution to Problem

The present inventors have conducted diligent research to solve the above problems.

As a result, the present inventors have found that when the number of substrates coated is large, simply defining a molecular weight range or molecular weight distribution does not allow an influence on wear resistance and resistance to chemical substances of a lubricating layer to be fully understood. Then, the present inventors have confirmed that, by defining molecular weight characteristics of a fluorine-containing ether compound contained in a lubricant for magnetic recording media in terms of a peak area ratio based on high-performance liquid chromatography, the wear resistance and resistance to chemical substances of the lubricating layer can be maintained without deteriorating even though the number of substrates coated is increased in a lubricant coating step, and thus have completed the present invention.

Configurations of the present invention are as follows: [1] A lubricant for magnetic recording media, comprising a fluorine-containing ether compound represented by formula (1), wherein a proportion of molecules with the same molecular weight contained in the fluorine-containing ether compound is 90% or more in terms of a peak area ratio based on high-performance liquid chromatography.

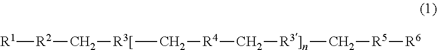

(wherein n is any one of integers of 0 to 2. R³ and R^{3′} are each independently a perfluoropolyether chain represented by formula (1f). When n is 1 or 2, R³ and one or two R^{3′}s are partially or totally the same, or are different from each other. R⁴ is a divalent linking group having 1 to 3 polar groups. When n is 2, two R⁴s are the same or different. R² and R⁵ are divalent linking groups having one or more polar groups, and are the same or different. An end of R² on a side bonded to R¹ is an oxygen atom. An end of R⁵ on a side bonded to R⁶ is an oxygen atom. R¹ and R⁶ are each a terminal group bonded to an oxygen atom at an end of R² or R⁵, and are the same or different. R¹ and R⁶ are hydrogen atoms or organic groups having 1 to 50 carbon atoms.).

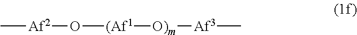

(wherein Af¹ represents a divalent perfluorohydrocarbon group having a linear or branched structure having 2 to 5 carbon atoms. When Af¹ has a linear structure, repeating units represented by (Af¹—O) are all the same. When Af¹ has a branched structure, a structure in which an orientation direction of Af¹ in the repeating unit represented by (Af¹—O) is interchanged from left to right is optionally partially included. Af² and Af³ each independently represent a divalent linear perfluorohydrocarbon group having 1 to 4 carbon atoms. m represents an average degree of polymerization and is a real number of 1 to 13.).
[2] A lubricant for magnetic recording media, comprising a fluorine-containing ether compound represented by formula (1), wherein a proportion of molecules represented by formula (1s) to 100% of the fluorine-containing ether compound represented by formula (1) is 90% or more in terms of a peak area ratio based on high-performance liquid chromatography.

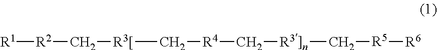

(wherein n is any one of integers 0 to 2. R³ and R^{3′} are each independently a perfluoropolyether chain represented by formula (1f). When n is 1 or 2, R³ and one or two R^{3′}s are partially or totally the same, or are different from each other. R⁴ is a divalent linking group having 1 to 3 polar groups. When n is 2, two R⁴s are the same or different. R² and R⁵ are divalent linking groups having one or more polar groups, and are the same or different. An end of R² on a side bonded to R¹ is an oxygen atom. An end of R⁵ on a side bonded to R⁶ is an oxygen atom. R¹ and R⁶ are each a terminal group bonded to an oxygen atom at an end of R² or R⁵, and are the same or different. R¹ and R⁶ are hydrogen atoms or organic groups having 1 to 50 carbon atoms.).

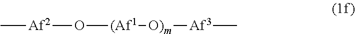

(wherein Af¹ represents a divalent perfluorohydrocarbon group having a linear or branched structure having 2 to 5 carbon atoms. When Af¹ has a linear structure, repeating units represented by (Af¹—O) are all the same. When Af¹ has a branched structure, a structure in which an orientation direction of Af¹ in the repeating unit represented by (Af¹—O) is interchanged from left to right is optionally partially included. Af² and Af³ each independently represent a divalent linear perfluorohydrocarbon group having 1 to 4 carbon atoms. m represents an average degree of polymerization and is a real number of 1 to 13.).

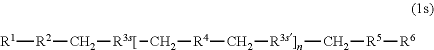

(wherein the same symbols as in formula (1) represent the same structures or values as in formula (1). R^{3s} and R^{3s′} are each independently a perfluoropolyether chain represented by formula (1f-1).).

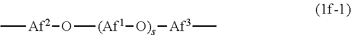

(wherein the same symbols as in formula (1f) represent the same structures as in formula (1f). s represents a degree of polymerization and is any one of integers of 1 to 13.). [3] The lubricant for magnetic recording media according to [1] or [2], wherein a number-average molecular weight of the fluorine-containing ether compound is 500 to 10,000.
[4] A method for producing a lubricant for magnetic recording media, the lubricant comprising a fluorine-containing ether compound represented by formula (1), wherein a proportion of molecules with the same molecular weight contained in the fluorine-containing ether compound is 90% or more in terms of a peak area ratio based on high-performance liquid chromatography,
the method comprising a step of synthesizing the fluorine-containing ether compound using a fluorinated polyalkylene glycol represented by formula (1f′) as a raw material,
wherein a proportion of molecules with the same molecular weight contained in the fluorinated polyalkylene glycol is 90% or more in terms of a peak area ratio based on high-performance liquid chromatography.

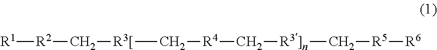

(wherein n is any one of integers of 0 to 2. R³ and R^{3′} are each independently a perfluoropolyether chain represented by formula (1f). When n is 1 or 2, R³ and one or two R^{3′}s are partially or totally the same, or are different from each other. R⁴ is a divalent linking group having 1 to 3 polar groups. When n is 2, two R⁴s are the same or different. R² and R⁵ are divalent linking groups having one or more polar groups, and are the same or different. An end of R² on a side bonded to R¹ is an oxygen atom. An end of R⁵ on a side bonded to R⁶ is an oxygen atom. R¹ and R⁶ are each a terminal group bonded to an oxygen atom at an end of R² or R⁵, and are the same or different. R¹ and R⁶ are hydrogen atoms or organic groups having 1 to 50 carbon atoms.).

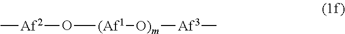

(wherein Af¹ represents a divalent perfluorohydrocarbon group having a linear or branched structure having 2 to 5 carbon atoms. When Af¹ has a linear structure, repeating units represented by (Af¹—O) are all the same. When Af¹ has a branched structure, a structure in which an orientation direction of Af¹ in the repeating unit represented by (Af¹—O) is interchanged from left to right is optionally partially included. Af² and Af³ each independently represent a divalent linear perfluorohydrocarbon group having 1 to 4 carbon atoms. m represents an average degree of polymerization and is a real number of 1 to 13.).

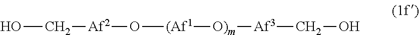

(wherein Af¹, Af², Af³ and m correspond to those in formula (1f). In formula (1), when n is 1 or 2 and when R³ and one or two R^{3′}s are not all the same, formula (1f′) represents a fluorine-containing polyalkylene glycol corresponding to each of R³ and R^{3′}.)
[5] The method for producing a lubricant for magnetic recording media according to [4], wherein the fluorinated polyalkylene glycol represented by formula (1f′) is synthesized by a method comprising the following steps (A) to (C):
step (A) of liquid-phase fluorinating a both-end-protected polyalkylene glycol represented by formula (2) in which a proportion of molecules with the same molecular weight is 90% or more in terms of a peak area ratio based on high-performance liquid chromatography;
step (B) of reacting the obtained fluorinated reactant represented by formula (3) with an alcohol having 1 to 6 carbon atoms; and
step (C) of carrying out a reduction reaction of the obtained both-end esterified product represented by formula (4).

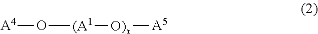

(wherein A¹ represents a divalent hydrocarbon group having a linear or branched structure having 2 to 5 carbon atoms. When A¹ has a linear structure, repeating units represented by (A¹—O) are all the same. When A¹ has a branched structure, a structure in which an orientation direction of A¹ in the repeating unit represented by (A¹—O) is interchanged from left to right is optionally partially included. x represents an average degree of polymerization and is a real number of 3 to 15. A⁴ and A⁵ each independently represent a protecting group for a hydroxy group.)

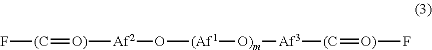

(wherein Af¹, Af², Af³ and m correspond to those in formula (1f).).

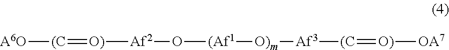

(wherein Af¹, Af², Af³ and m correspond to those in formula (1f). A⁶ and A⁷ each independently represent an alkyl group having 1 to 6 carbon atoms.).
[6] The method for producing a lubricant for magnetic recording media according to [5], comprising, prior to the step (A), a polyether synthesis step of carrying out a nucleophilic substitution reaction of reacting two or more compounds having a polyether chain or a monomer unit constituting the polyether chain to synthesize a polyether compound having a repeating unit represented by (A¹—O).
[7] A method for producing a lubricant for magnetic recording media, the lubricant comprising a fluorine-containing ether compound represented by formula (1), wherein a proportion of molecules represented by formula (1s) to 100% of the fluorine-containing ether compound represented by formula (1) is 90% or more in terms of a peak area ratio based on high-performance liquid chromatography,
the method comprising a step of synthesizing the fluorine-containing ether compound using a fluorinated polyalkylene glycol represented by formula (1f′) as a raw material,
wherein a proportion of molecules represented by formula (1f′-1) to 100% of the fluorinated polyalkylene glycol represented by formula (1f′) is 90% or more in terms of a peak area ratio based on high-performance liquid

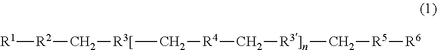

(wherein n is any one of integers of 0 to 2. R³ and R^{3′} are each independently a perfluoropolyether chain represented by formula (1f). when n is 1 or 2, R³ and one or two R^{3′}s are partially or totally the same, or are different from each other. R⁴ is a divalent linking group having 1 to 3 polar groups. when n is 2, two R⁴s are the same or different. R² and R⁵ are divalent linking groups having one or more polar groups, and are the same or different. An end of R² on a side bonded to R¹ is an oxygen atom. An end of R⁵ on a side bonded to R⁶ is an oxygen atom. R¹ and R⁶ are each a terminal group bonded to an oxygen atom at an end of R² or R⁵, and are the same or different. R¹ and R⁶ are hydrogen atoms or organic groups having 1 to 50 carbon atoms.).

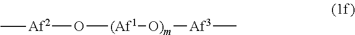

(wherein Af¹ represents a divalent perfluorohydrocarbon group having a linear or branched structure having 2 to 5 carbon atoms. When Af¹ has a linear structure, repeating units represented by (Af¹—O) are all the same. When Af¹ has a branched structure, a structure in which an orientation direction of Af¹ in the repeating unit represented by (Af¹—O) is interchanged from left to right is optionally partially included. Af² and Af³ each independently represent a divalent linear perfluorohydrocarbon group having 1 to 4 carbon atoms. m represents an average degree of polymerization and is a real number of 1 to 13.).

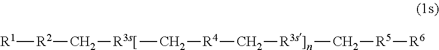

(wherein the same symbols as in formula (1) represent the same structures or values as in formula (1). R^{3s} and R^{3s′} are each independently a perfluoropolyether chain represented by formula (1f-1).).

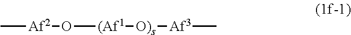

(wherein the same symbols as in formula (1f) represent the same structures as in formula (1f). s represents a degree of polymerization and is any one of integers of 1 to 13.).

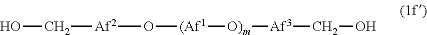

(wherein Af¹, Af², Af³ and m correspond to those in formula (1f). In formula (1), when n is 1 or 2 and when R³ and one or two R^{3′}s are not all the same, formula (1f′) represents a fluorine-containing polyalkylene glycol corresponding to each of R³ and R^{3′}.)

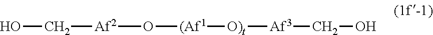

(wherein the same symbols as in formula (1f′) represent the same structures as in formula (1f′). t represents a degree of polymerization and is any one of integers of 1 to 13.). [8] The method for producing a lubricant for magnetic recording media according to [7], wherein the fluorinated polyalkylene glycol represented by formula (1f′) is synthesized by a method comprising the following steps (A-1) to (C-1):
step (A-1) of liquid-phase fluorinating a both-end-protected polyalkylene glycol represented by formula (2), wherein a proportion of molecules represented by formula (2-1) is 90% or more in terms of a peak area ratio based on high-performance liquid chromatography;
step (B-1) of reacting the obtained fluorinated reactant represented by formula (3) with an alcohol having 1 to 6 carbon atoms; and
step (C-1) of carrying out a reduction reaction of the obtained both-end esterified product represented by formula (4).

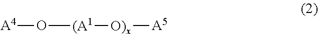

(wherein A¹ represents a divalent hydrocarbon group having a linear or branched structure having 2 to 5 carbon atoms. When A¹ has a linear structure, repeating units represented by (A¹—O) are all the same. When A¹ has a branched structure, a structure in which an orientation direction of A¹ in the repeating unit represented by (A¹—O) is interchanged from left to right is optionally partially included. x represents an average degree of polymerization and is a real number of 3 to 15. A⁴ and A⁵ each independently represent a protecting group for a hydroxy group.)

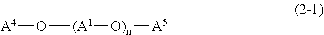

(wherein the same symbols as in formula (2) represent the same structures as in formula (2). u represents a degree of polymerization and is any one of integers of 3 to 15.).

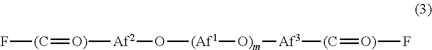

(wherein Af¹, Af², Af³ and m correspond to those in formula (1f).).

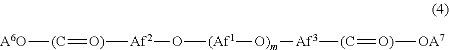

(wherein Af¹, Af², Af³ and m correspond to those in formula (1f). A⁶ and A⁷ each independently represent an alkyl group having 1 to 6 carbon atoms.).
[9] The method for producing a lubricant for magnetic recording media according to [8], comprising, prior to the step (A-1), a polyether synthesis step of carrying out a nucleophilic substitution reaction of reacting two or more compounds having a polyether chain or a monomer unit constituting the polyether chain to synthesize a polyether compound having a repeating unit represented by (A¹—O).
[10] The method for producing a lubricant for magnetic recording media according to [6] or [9],
wherein the polyether synthesis step comprises a step of reacting a compound having a leaving group at one end of a polyether chain or a monomer unit constituting the polyether chain and a protected hydroxy group at the other end
with a compound having a hydroxy group at one end of a polyether chain or a monomer unit constituting the polyether chain and a protected hydroxy group at the other end.
   [11] The method for producing a lubricant for magnetic recording media according to [6] or [9],
wherein the polyether synthesis step comprises a step of reacting a compound having a leaving group at one end of a polyether chain or a monomer unit constituting the polyether chain and a protected hydroxy group at the other end
with a compound having hydroxy groups at both ends of a polyether chain or a monomer unit constituting the polyether chain.
   [12] The method for producing a lubricant for magnetic recording media according to [6] or [9],
wherein the polyether synthesis step comprises a step of reacting a compound having a hydroxy group at one end of a polyether chain or a monomer unit constituting the polyether chain and a protected hydroxy group at the other end
with a compound having leaving groups at both ends of a polyether chain or a monomer unit constituting the polyether chain.
   [13] A magnetic recording medium, comprising at least a magnetic layer, a protective layer, and a lubricating layer sequentially provided on a substrate, wherein the lubricating layer comprises the lubricant for magnetic recording media according to any one of [1] to [3].
   [14] A method for producing a magnetic recording medium, comprising a step of coating a surface of a protective layer in a substrate on which at least a magnetic layer and the protective layer are formed, with the lubricant for magnetic recording media according to any one of [1] to [3] to form a lubricating layer.

### Advantageous Effects of Invention

The lubricant for magnetic recording media of the present invention can maintain wear resistance and resistance to chemical substances of a lubricating layer formed in a lubricant coating step even though the number of substrates coated therein is increased. According to the method for producing a lubricant for magnetic recording media of the present invention, such a lubricant for magnetic recording media can be efficiently produced.

Since the wear resistance and resistance to chemical substances of the lubricating layer can be highly maintained upon its production, the magnetic recording medium of the present invention has excellent reliability and durability.

By the method for producing a magnetic recording medium of the present invention, such a magnetic recording medium can be efficiently produced.

### BRIEF DESCRIPTION OF DRAWINGS

FIG. 1 is a schematic cross-sectional view illustrating a preferred embodiment of the magnetic recording medium of the present invention.

FIG. 2 is a ¹H-NMR spectrum of a 1,3-propanediol pentamer synthesized in Example 1.

FIG. 3 is a ¹H-NMR spectrum of a both-end-acetylated 1,3-propanediol pentamer synthesized in Example 1.

FIG. 4 is a GPC chromatogram of a both-end-acetylated 1,3-propanediol pentamer synthesized in Example 1.

FIG. 5 is a ¹⁹F-NMR spectrum of a fluorinated 1,3-propanediol pentamer synthesized in Example 1.

FIG. 6 is a ¹H-NMR spectrum of a compound (A¹) synthesized in Example 1.

FIG. 7 is a ¹⁹F-NMR spectrum of a compound (A¹) synthesized in Example 1.

FIG. 8 is a normal-phase LC chromatogram of a compound (A¹) synthesized in Example 1.

FIG. 9 is a GPC chromatogram of a both-end-acetylated 1,3-propanediol polymer synthesized in Comparative Example 1.

FIG. 10 is a GPC chromatogram of a both-end-acetylated 1,3-propanediol polymer after molecular weight distribution adjustment, synthesized in Comparative Example 1.

FIG. 11 is a GPC chromatogram of a fluorinated 1,3-propanediol polymer synthesized in Comparative Example 1.

FIG. 12 is a ¹H-NMR spectrum of a compound (B1) synthesized in Comparative Example 1.

FIG. 13 is a ¹⁹F-NMR spectrum of a compound (B1) synthesized in Comparative Example 1.

FIG. 14 is a normal-phase LC chromatogram of a compound (B1) synthesized in Comparative Example 1.

FIG. 15 is a view comparing normal phase LC chromatograms of compounds of (A¹) to (C1).

FIG. 16 is a graph illustrating the results of a wear resistance test for compounds (A¹) to (C1).

FIG. 17 is a graph illustrating the results of a test for resistance to chemical substances for compounds (A¹) to (C1)

### DESCRIPTION OF EMBODIMENT

Embodiments of the lubricant for magnetic recording media of the present invention will be described in detail below. Incidentally, the present invention is not limited to embodiments shown below. The present invention allows for addition, omission, substitution, and modification of the number, amount, ratio, material, and configuration, for example, within the ranges that do not deviate from the gist of the present invention.

The lubricant of the present embodiment contains a specific fluorine-containing ether compound. First, the fluorine-containing ether compound will be described.

### [Fluorine-Containing Ether Compound]

The fluorine-containing ether compound is represented by the following formula (1), and is composed of substantially the same components, and when a proportion of a molecule with the same molecular weight (molecule constituting the same component) is defined in terms of a peak area ratio based on high-performance liquid chromatography, it is 90% or more, preferably 93% or more, and more preferably 95% or more. Note that the upper limit is 100%. Being 90% or more in terms of a peak area ratio based on high-performance liquid chromatography means that constituent elements constituting a main chain, the number of repetitions, a side chain, and a terminal functional group of a compound to be measured are substantially the same, and the closer the peak area ratio is to 100%, the smaller the variation in the number of repetitions becomes, and when the peak area ratio is 100%, the number of repetitions is also the same. This definition also applies to raw materials and intermediate products when synthesizing a fluorine-containing ether compound. It is noted that when the compound to be measured contains an isomer (for example, when an isomer is created in the course of synthesis of the compound), a molecule with the same molecular weight also contains an isomer.

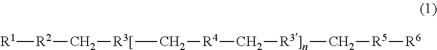

(wherein n is any one of integers of 0 to 2. R³ and R^{3′} are each independently a perfluoropolyether chain represented by formula (1f). When n is 1 or 2, R³ and one or two R^{3′}s are partially or totally the same, or are different from each other. R⁴ is a divalent linking group having 1 to 3 polar groups. When n is 2, two R⁴s are the same or different. R² and R⁵ are divalent linking groups having one or more polar groups, and are the same or different. An end of R² on a side bonded to R¹ is an oxygen atom. An end of R⁵ on a side bonded to R⁶ is an oxygen atom. R¹ and R⁶ are each a terminal group bonded to an oxygen atom at an end of R² or R⁵, and are the same or different. R¹ and R⁶ are hydrogen atoms or organic groups having 1 to 50 carbon atoms.).

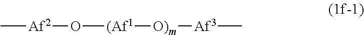

(wherein Af¹ represents a divalent perfluorohydrocarbon group having a linear or branched structure having 2 to 5 carbon atoms. When Af¹ has a linear structure, repeating units represented by (Af¹—O) are all the same. When Af¹ has a branched structure, a structure in which an orientation direction of Af¹ in the repeating unit represented by (Af¹—O) is interchanged from left to right is optionally partially included. Af² and Af³ each independently represent a divalent linear perfluorohydrocarbon group having 1 to 4 carbon atoms. m represents an average degree of polymerization and is a real number of 1 to 13.).
PFPE Chain Represented by R³ and R^{3′}

In the fluorine-containing ether compound of the present embodiment represented by formula (1), R³ and R^{3′} are each independently the PFPE chain represented by formula (1f). When a surface of a protective layer is coated with a lubricant containing the fluorine-containing ether compound to form a lubricating layer, the PFPE chain covers the surface of the protective layer and imparts lubricity to the lubricating layer to reduce frictional force between a magnetic head and the protective layer.

R³ and one or two R^{3′}s are partially or totally the same, or are different from each other. R³ and one or two R^{3′}s are all preferably the same. This is because a covering state of the fluorine-containing ether compound on the protective layer becomes more uniform, resulting in a lubricating layer having more favorable adhesiveness. R³ and one or two R^{3′}s being the same also includes the case where a repeating unit structure of the PFPE chain is the same but an average degree of polymerization is different.

In formula (1f), Af¹ represents a divalent perfluorohydrocarbon group having a linear or branched structure and having 2 to 5 carbon atoms. Specific examples of Af¹ include, for example, —CF₂—CF₂—, —CF₂—CF₂—CF₂—, —CF₂—CF(CF₃)—, —CF(CF₃)—CF₂—, —CF₂—CF₂—CF₂—CF₂—, —CF(CF₃)—CF₂—CF₂—, —CF₂—CF(CF₃)—CF₂—, —CF₂—CF₂—CF(CF₃)—, —CF₂—CF₂—CF₂—CF₂—CF₂—, —CF(CF₃)—CF₂—CF₂—CF₂—, —CF₂—CF(CF₃)—CF₂—CF₂—, —CF₂—CF₂—CF(CF₃)—CF₂—, —CF₂—CF₂—CF₂—CF(CF₃)—, and —CF(CF₃)—CF₂—CF(CF₃)—. Af¹ is preferably selected from —CF₂—CF₂—, —CF₂—CF₂—CF₂—, —CF₂—CF(CF₃)—, —CF(CF₃)—CF₂—, —CF₂—CF₂—CF₂—CF₂—, —CF₂—CF₂—CF₂—CF₂—CF₂—, and —CF(CF₃)—CF₂—CF(CF₃)—. Af¹ is more preferably —CF₂—CF₂—, —CF₂—CF₂—CF₂—, —CF₂—CF(CF₃)—, —CF(CF₃)—CF₂—, or —CF₂—CF₂—CF₂—CF₂—, and most preferably —CF₂—CF₂— or —CF₂—CF₂—CF₂—.

In formula (1f), when Af¹ has a linear structure, Af's in repeating units represented by (Af¹—O) are all the same, and when Af¹ has a branched structure, a structure in which an orientation direction of Af¹ in each repeating unit represented by (Af¹—O) is interchanged from left to right is optionally partially included. For example, when Af¹ has a branched structure, a bonding manner between each repeating unit represented by (Af¹—O) is not limited to a head-to-tail bonding structure, and may include a head-to-head bonding structure and tail-to-tail bonding structure.

In formula (1f), Af² and Af³ each independently represent a divalent linear perfluorohydrocarbon group having 1 to 4 carbon atoms. Af² and Af³ are each determined according to a structure of the repeating unit represented by (Af¹—O).

Specifically, R³ and R^{3′} in formula (1) are each preferably independently any one of the following formulae (1f-a) to (1f-d).

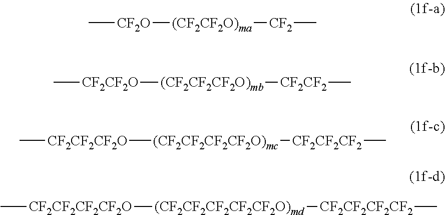

(In formulae (1f-a) to (1f-d), ma to md each represents an average degree of polymerization and is a real number of 1 to 13.).

In formula (1f-a), ma indicating an average degree of polymerization represents a real number of 1 to 13. When ma is 1 to 13, the number-average molecular weight of the fluorine-containing ether compound of the present embodiment tends to fall in a preferred range. ma is preferably 3 to 11 and more preferably 4 to 10.

In formula (1f-b), mb indicating an average degree of polymerization is a real number of 1 to 13. When mb is 1 to 13, the number-average molecular weight of the fluorine-containing ether compound of the present embodiment tends to fall in a preferred range. mb is preferably 1 to 8 and more preferably 2 to 6.

In formula (1f-c), mc indicating an average degree of polymerization, is a real number of 1 to 13. When mc is 1 to 13, the number-average molecular weight of the fluorine-containing ether compound of the present embodiment tends to fall in a preferred range. mc is preferably 1 to 6 and more preferably 2 to 4.

In formula (1f-d), md indicating an average degree of polymerization is a real number of 1 to 13. When md is 1 to 13, the number-average molecular weight of the fluorine-containing ether compound of the present embodiment tends to fall in a preferred range. md is preferably 1 to 5 and more preferably 1 to 3.

When R³ and R^{3′} in formula (1) are each any one of formulae (1f-a) to (1f-d), a fluorine-containing ether compound providing a lubricating layer having favorable lubricity is obtained. When R³ and R^{3′} are each any one type of PFPE chains represented by formulae (1f-a) to (1f-d), a proportion of the number of oxygen atoms (the number of ether bonds (—O—)) to the number of carbon atoms in a PFPE chain is appropriate. This results in a fluorine-containing ether compound with appropriate hardness. Therefore, the fluorine-containing ether compound, with which a surface of a protective layer is coated, is less likely to aggregate on the protective layer, making it possible to form an even thinner lubricating layer with a sufficient coverage. Also, the fluorine-containing ether compound having appropriate flexibility enables forming a lubricating layer with more favorable resistance to chemical substances and wear resistance.

### Divalent Linking Group Represented by R⁴

In the fluorine-containing ether compound represented by formula (1), R⁴ is a divalent linking group having 1 to 3 polar groups. When n is 1 or 2, R⁴ is each arranged between PFPE chains represented by R³ and one or two R^{3′}s. This allows R⁴ to bring the fluorine-containing ether compound and the protective layer into close contact to form a thin lubricating layer with a sufficient coverage.

When the number n of the repeating unit [—CH₂—R⁴—CH₂—R^{3′}]in formula (1) is 2, two R⁴s are the same or different. When the two R⁴s are the same, a covering state of the fluorine-containing ether compound on the protective layer becomes more uniform, enabling forming a lubricating layer having more favorable adhesiveness.

When n is 2, the phrase “two R⁴s are the same” used herein means that, of two R^{3′}s of the fluorine-containing ether compound, atoms contained in the two R⁴s have the same configuration and are arranged symmetrically with respect to R^{3′} arranged at the center of the fluorine-containing ether compound represented by formula (1).

The divalent linking group represented by R⁴ preferably has oxygen atoms arranged at both ends. The oxygen atoms arranged at both ends of the linking group form ether bonds (—O—) with methylene groups (—CH₂—) arranged on both sides of R⁴. These two ether bonds impart appropriate flexibility to the fluorine-containing ether compound represented by formula (1) and increase affinity between a polar group of the divalent linking group represented by R⁴ and a protective layer.

The number of carbon atoms contained in R⁴ is preferably 3 to 9 and more preferably 3 to 6.

The number of polar groups of R⁴ is 1 to 3 and is preferably 1 or 2 and more preferably 1. When the number of polar groups contained in R⁴ is two or more, the types of the polar groups are partially or totally the same, or are different from each other.

Examples of the polar group in R⁴ includes, for example, a hydroxy group (—OH), an amino group (—NH₂), a carboxyl group (—COOH), an aldehyde group (—COH), a carbonyl group (—CO—), and a sulfonic acid group (—SO₃H). Among these, the polar group is a particularly preferable hydroxy group. The hydroxy group has a strong interaction with a protective layer, particularly a protective layer formed of a carbon-based material. For this reason, when the polar group of R⁴ contains the hydroxy group, a lubricating layer containing the fluorine-containing ether compound has even higher adhesiveness to the protective layer. When n is 2, at least one of the polar groups of each of the two R⁴s preferably contains the hydroxy group, and both polar groups more preferably contain the hydroxy group.

Specifically, R⁴ is preferably represented by the following formula (1-1). In formula (1-1), the oxygen atom on the left side is bonded to the methylene group on the R² side of the two methylene groups bonded to R⁴, and the oxygen atom on the right side is bonded to the methylene group on the R⁵ side. When R⁴ is represented by formula (1-1), the fluorine-containing ether compound represented by formula (1) is more easily synthesized, which is preferable.

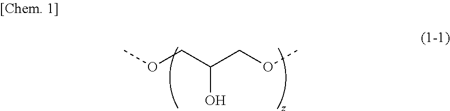

(In formula (1-1), z is an integer of 1 to 3.)

In formula (1-1), z is an integer of 1 to 3, preferably an integer of 1 to 2 and most preferably 1. When z is 1, the number of hydroxy groups in the fluorine-containing ether compound is not too large, rendering it favorably hydrophobic. As a result, water which causes corrosion of a magnetic recording medium, can be effectively prevented from being intruded, and a lubricating layer comes to have a high corrosion inhibition effect on the magnetic recording medium.

Note, however, the chain line used herein in the formula indicates a bonded end with other structures.

Divalent Linking Groups Represented by R² and R⁵

In the fluorine-containing ether compound represented by formula (1), R² and R⁵ are divalent linking groups having one or more polar groups. R² has an oxygen atom at the end on a side bonded to R¹, and when R¹ is an organic group, it is bonded to R¹ by an ether bond. R⁵ has an oxygen atom at the end on a side bonded to R⁶, and when R⁶ is an organic group, it is bonded to R⁶ by ether bond. In the fluorine-containing ether compound represented by formula (1), R² and R⁵ each has one or more polar groups, so that when a lubricant containing this is used to form a lubricating layer on a protective layer, a suitable interaction between the lubricating layer and the protective layer develops. R² and R⁵ can be appropriately selected depending on, for example, performance which is required for a lubricant containing the fluorine-containing ether compound.

R² and R⁵ are the same or different. When R² and R⁵ are the same, a covering state of the fluorine-containing ether compound on the protective layer becomes more uniform, enabling forming a lubricating layer having more favorable adhesiveness.

The phrase “R² and R⁵ are the same” means that an atom contained in R² and an atom contained in R⁵ have the same configuration and are symmetrically arranged with respect to a skeleton (—R³[—CH₂—R⁴—CH₂—R^{3′}]ₙ—) arranged at the center of the fluorine-containing ether compound represented by formula (1).

The numbers of carbon atoms contained in R² and R⁵ are each preferably 3 to 9 and more preferably 3 to 6.

The numbers of polar groups of R² and R⁵ are each 1 or more, preferably 1 to 3, and more preferably 1 or 2. When the number of polar groups in R² and/or R⁵ is 2 or more, the types of polar groups are partially or totally the same, or are different from each other.

Examples of the polar groups of R² and R⁵ include, for example, a hydroxy group (—OH), an amino group (—NH₂), a carboxy group (—COOH), an aldehyde group (—COH), a carbonyl group (—CO—), and a sulfonic acid group (—SO₃H). Among these, the polar group is a particularly preferable hydroxy group.

The hydroxy group has a strong interaction with a protective layer, particularly a protective layer formed of a carbon-based material. For this reason, when at least a moiety of the polar groups in R² and/or R⁵ is the hydroxy group, a lubricating layer containing the fluorine-containing ether compound has even higher adhesiveness to the protective layer. In the present embodiment, the polar groups of R² and R⁵ are all preferably hydroxy groups.

When the polar groups of R² and/or R⁵ contain the hydroxy group, the total number of a hydroxy group contained in R² and a hydroxy group contained in R⁵ in formula (1) is preferably 2 to 6, more preferably 2 to 4, and most preferably 2. When the total number of the above-described hydroxy groups is 2 or more, interactions between the hydroxy groups of R² and R⁵ and a protective layer are effectively obtained. As a result, a fluorine-containing ether compound capable of forming a lubricating layer that has high adhesiveness to a protective layer is obtained. Also, when the total number of the above-described hydroxy groups is 6 or less, the number of polar groups that are not involved in a bond between the lubricating layer and an active site on the protective layer is reduced. Therefore, the polar groups that are not involved in the bond between the lubricating layer and the active site on the protective layer can prevent environmental substances that create pollutants and water that causes corrosion of magnetic recording media from being attracted to the lubricating layer. This makes it possible to form a lubricating layer capable of more effectively inhibiting contamination and corrosion of magnetic recording media. Also, when the total number of the above hydroxy groups is 4 or less, flowability of the lubricating layer containing the fluorine-containing ether compound becomes sufficiently high. For this reason, even though the lubricating layer containing the fluorine-containing ether compound is partially deformed due to wear, and the fluorine-containing ether compound in the lubricating layer moves to another location, the lubricating layer has a high ability to restore itself to its original position, giving superior wear resistance.

It is preferable that the divalent linking group represented by R² has an oxygen atom at the end of R² on a side bonded to R¹, and also has an oxygen atom arranged at the other end (the end bonded to CH₂ adjacent to R²). It is also preferable that the divalent linking group represented by R⁵ has an oxygen atom at the end of R⁵ on a side bonded to R⁶, and also has an oxygen atom arranged at the other end (the end bonded to CH₂ adjacent to R⁵). When R¹ and R⁶ are organic groups, oxygen atoms at both ends of the divalent linking groups represented by R² and R⁵ form ether bonds (—O—) with atoms bonded to both sides thereof. These ether bonds impart appropriate flexibility to the fluorine-containing ether compound represented by formula (1) and increase affinity between polar groups of the divalent linking groups represented by R² and R⁵ and a protective layer.

In the fluorine-containing ether compound represented by formula (1), —R²— is preferably represented by —[B]-[A]-O—, and —R⁵— is preferably represented by —O—[C]-[D]-. That is, the fluorine-containing ether compound of the present embodiment is preferably represented by the following formula (1′).

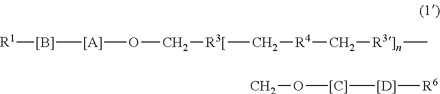

(wherein n, R¹, R³, R^{3′}, R⁴ and R⁶ are the same as in formula (1). In formula (1′), [A] is represented by the following formula (1-2). a in formula (1-2) is an integer of 0 to 3. In formula (1′), [B] is represented by the following formula (1-3). b in formula (1-3) is an integer of 0 to 3, and c is an integer of 2 to 5, with the proviso that the sum of a in formula (1-2) and b in formula (1-3) is 1 to 3. In formula (1′), [A] and [B] may be interchanged. In formula (1′), [C]is represented by the following formula (1-4). d in formula (1-4) is an integer of 0 to 3. In formula (1′), [D] is represented by the following formula (1-5). e in formula (1-5) is an integer of 0 to 3, and f is an integer of 2 to 5, with the proviso that the sum of d in formula (1-4) and e in formula (1-5) is 1 to 3. In formula (1′), [C] and [D] may be interchanged.).

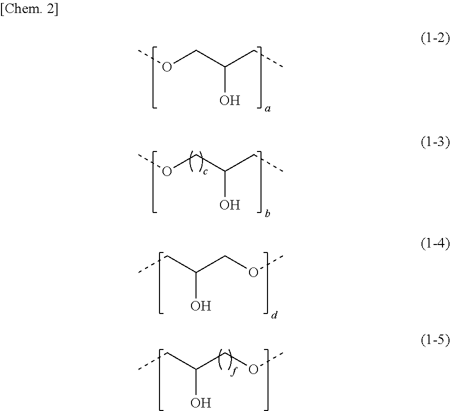

### Linking Group Represented by —[B]-[A]-O—

In the fluorine-containing ether compound of the present embodiment represented by formula (1′), [A] is represented by the above formula (1-2), and [B] is represented by the above formula (1-3). [A] and [B] in formula (1′) are divalent linking groups. In formula (1′), [A] and [B] may be interchanged. a in formula (1-2) and b in formula (1-3) are integers of 0 to 3, provided that the sum of the values of a and b is 1 to 3.

From the viewpoint of availability of raw materials and facilitation of synthesis, a combination of a being 1 and b being 0, or a combination of a being 0 and b being 1 is preferable in formula (1-2) and formula (1-3).

Furthermore, from the viewpoint of adhesiveness to a protective layer, a combination of a being 2 and b being 0, or a combination of a being 1 and b being 1 is preferable in formula (1-2) and formula (1-3). In particular, when a is 2 and b is 0, in the fluorine-containing ether compound, a direction formed by the two hydroxy groups in formula (1-2) arranged is three-dimensionally the same direction as an extension direction of a PFPE chain, resulting in a tendency to facilitate the two hydroxy groups in formula (1-2) to be adsorbed to a protective layer. Also, when a and b are 1 and the bonding order of [A] and [B] is —[A]-[B]—O— from the R¹ side, a distance between hydroxy groups contained in the —[A]-[B]— structure becomes farther. This makes it possible to decline intramolecular hydrogen bonding of the fluorine-containing ether compound represented by formula (1′) and to enhance adhesiveness to a protective layer.

c in formula (1-3) is an integer of 2 to 5. When b is an integer of 1 to 3, c is preferably an integer of 2 to 4 and most preferably 2.

In formula (1′), when [A] is directly bonded to R¹, it is preferable that R¹ is not a hydrogen atom (R¹ is an organic group having 1 to 50 carbon atoms.). In this case, a carbon atom to which a hydroxy group in —[B]-[A]-O— is bonded is not directly bonded to a carbon atom to which the adjacent hydroxy group is bonded. As a result, the hydroxy group in —[B]-[A]-O— is easily adsorbed to a protective layer.

### Linking Group Represented by —O—[C]-[D]—

In the fluorine-containing ether compound of the present embodiment represented by formula (1′), [C] is represented by the above-described formula (1-4) and [D] is represented by the above formula (1-5). [C] and [D] in formula (1′) are divalent linking groups. In formula (1′), [C] and [D] may be interchanged. d in formula (1-4) and e in formula (1-5) are integers of 0 to 3, provided that the sum of the values of d and e is 1 to 3.

From the viewpoint of availability of raw materials and facilitation of synthesis, a combination of d being 1 and e being 0, or a combination of d being 0 and e being 1, is preferred in formula (1-4) and formula (1-5).

From the viewpoint of adhesiveness to a protective layer, a combination of d of 2 and e of 0, or a combination of d of 1 and e of 1 is preferable in formula (1-4) and formula (1-5). Particularly, when d is 2 and e is 0, in the fluorine-containing ether compound, a direction formed by the two hydroxy groups of formula (1-4) arranged is three-dimensionally the same direction as an extension direction of a PFPE chain, resulting in a tendency of facilitating the two hydroxy groups in formula (1-4) to be adsorbed to a protective layer. Also, when d and e are 1 and the bonding order of [C] and [D] is —O-[D]-[C]— from the R³ side, a distance between hydroxy groups contained in the -[D]-[C]-structure becomes farther. This makes it possible to lower intramolecular hydrogen bonding force of the fluorine-containing ether compound represented by formula (1′) and to enhance adhesiveness to a protective layer.

In formula (1-5), f is an integer of 2 to 5. When e is an integer of 1 to 3, f is preferably an integer of 2 to 4 and most preferably 2.

In formula (1′), when [C] is directly bonded to R⁶, it is preferable that R⁶ is not a hydrogen atom (R⁶ is an organic group having 1 to 50 carbon atoms.). In this case, a carbon atom to which a hydroxy group contained in —O—[C]-[D]— is bonded is not directly bonded to a carbon atom to which the adjacent hydroxy group is bonded. As a result, the hydroxy group in —O—[C]-[D]— is more likely to be adsorbed to a protective layer.

End Groups Represented by R¹ and R⁶

In the fluorine-containing ether compound represented by formula (1), the end groups represented by R¹ and R⁶ are each independently a hydrogen atom or an organic group having 1 to 50 carbon atoms. R¹ and R⁶ are the same or different. R¹ and R⁶ can be appropriately selected depending on, for example, performance required for a lubricant containing the fluorine-containing ether compound.

The terminal groups represented by R¹ and R⁶ are each preferably independently any one of the following formulae (1-6), (1-7) and (1-8). In this case, R¹ and R⁶ having large molecular weights enable reducing a proportion of fluorine atoms in a fluorine-containing ether compound molecule and inhibiting the surface free energy of the entire molecule from increasing.

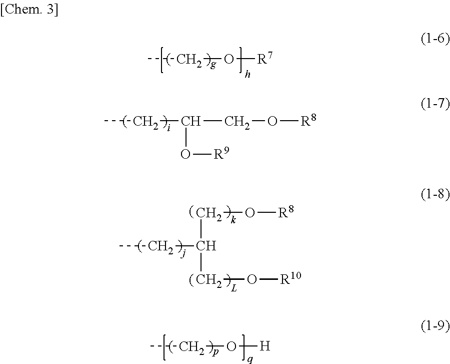

(In formula (1-6), g represents an integer of 2 to 6, and h represents 0 or 1. R⁷ is any one of a hydrogen atom, an alkyl group which may have a substituent not including a hydroxy group, and an organic group having at least one double bond or triple bond, provided that the alkyl group and the organic group may be linear or branched.)
(In formula (1-7), i represents an integer of 1 to 6. R⁸ and R⁹ are represented by formula (1-9). R⁸ and R⁹ are the same or different.).
(In formula (1-8), j represents an integer of 0 to 6. k and L each independently represent an integer of 1 to 6. R¹⁰ and R¹¹ are hydrogen atoms or represented by formula (1-9). R¹⁰ and R¹¹ are the same or different.).
(In formula (1-9), p represents an integer of 2 to 6, and q represents 1 or 2.)

### End Group Represented by Formula (1-6)

In formula (1-6), g represents an integer of 2 to 6, and h represents 0 or 1. When h in formula (1-6) is 0, a proportion of a fluorine atom in a fluorine-containing ether compound molecule decreases, making it possible to more effectively inhibit the surface free energy of the entire molecule from increasing. When h is 1, an ether bond contained in formula (1-6) imparts flexibility to the fluorine-containing ether compound represented by formula (1), making the compound more likely to be adsorbed to a protective layer. g is preferably an integer of 2 to 4 and more preferably 2 or 3. When g is 2 or 3, a proportion of fluorine atoms in a fluorine-containing ether compound molecule decreases, enabling inhibiting the surface free energy of the entire molecule from increasing.

R⁷ in formula (1-6) is any one of a hydrogen atom, an alkyl group which may have a substituent not containing a hydroxy group, and an organic group having at least one double bond or triple bond. When R⁷ is a hydrogen atom, R⁷ forms a hydroxy group together with the adjacent oxygen atom. When h in formula (1-6) is 1 and R⁷ is a hydrogen atom, R¹ and/or R⁶ represented by formula (1-6) are alkyl groups having a hydroxy group at their ends.

When R⁷ is a hydrogen atom and h in formula (1-6) is 1, preferred specific examples of R¹ and/or R⁶ represented by formula (1-6) include —CH₂CH₂—OH (g in formula (1-6) is 2) and —CH₂CH₂CH₂—OH (g in formula (1-6) is 3).

When R⁷ is an alkyl group which may have a substituent not containing a hydroxy group, it is preferably an alkyl group having 1 to 6 carbon atoms not containing a substituent, or an alkyl group having 1 to 6 carbon atoms and having a substituent. The substituent of the alkyl group having 1 to 6 carbon atoms and having a substituent is preferably a fluoro group, a cyano group, or a group having an amide bond, and does not contain a hydroxy group. The alkyl group having 1 to 6 carbon atoms and having a substituent is a group in which one or more hydrogen atoms of the alkyl group have been substituted with substituents, and may be a group in which all hydrogen atoms of the alkyl group have been substituted with substituents.

The alkyl group having 1 to 6 carbon atoms not containing a substituent and the alkyl group of the alkyl group having 1 to 6 carbon atoms and having a substituent may be linear or branched. Specific examples of the alkyl groups include a methyl group, an ethyl group, an n-propyl group, an isopropyl group, an n-butyl group, a sec-butyl group, an isobutyl group, a tert-butyl group, an n-pentyl group and a structural isomer thereof, and an n-hexyl group and a structural isomer thereof.

Examples of alkyl groups having 1 to 6 carbon atoms in which one or more hydrogen atoms have been substituted with fluoro groups include, for example, a trifluoromethyl group, a perfluoroethyl group, a perfluoropropyl group, a perfluorobutyl group, a perfluoropentyl group, a perfluorohexyl group, a 2,2,2-trifluoroethyl group, a 2,2,3,3,3-pentafluoropropyl group, a 2,2,2,2,2,2-hexafluoroisopropyl group, a 2,2,3,3,4,4,4-heptafluorobutyl group, a 2,2,3,3,4,4,5,5,5-nonafluoropentyl group, and a 2,2,3,3,4,4,5,5,6,6,6-undecafluorohexyl group.

The number of cyano groups in an alkyl group having 1 to 6 carbon atoms in which one or more hydrogen atoms have been substituted with cyano groups may be one or more than one. The large number of cyano groups renders too high polarity of a fluorine-containing ether compound, therefore the number of cyano groups is preferably 2 or less and most preferably 1.

Examples of the alkyl groups having 1 to 6 carbon atoms in which one or more hydrogen atoms have been substituted with cyano groups include, for example, a 2-cyanoethyl group, a 3-cyanopropyl group, a 4-cyanobutyl group, a 5-cyanopentyl group, a 6-cyanohexyl group, a 2-cyano-1-methylethyl group, and a 2,2′-dicyanoisopropyl group.

The number of amide bonds in an alkyl group having 1 to 6 carbon atoms in which one or more hydrogen atoms have been substituted with groups having an amide bond may be one or more than one. The large number of amide bonds renders too high polarity of the fluorine-containing ether compound, therefore the number of amide bonds is preferably two or less and most preferably one.

In the alkyl group having 1 to 6 carbon atoms in which one or more hydrogen atoms have been substituted with groups having an amide bond, a carbon atom of the alkyl group may be bonded to a carbon atom of the amide bond (—NR¹²C(═O)—; R¹² is a hydrogen atom or an organic group) or to a nitrogen atom of the amide bond. The bond of the amide bond (—NR¹²C(═O)—; R¹² is a hydrogen atom or an organic group) on the side not bonded to an alkyl group (end side) is preferably bonded to, for example, a hydrogen atom, a methyl group, an ethyl group, a propyl group, a butyl group, or a trifluoromethyl group, and is preferably bonded to a hydrogen atom or a methyl group. When R¹² in the above formula is an organic group, examples of R¹² include a methyl group, an ethyl group, a propyl group, and a butyl group.

Specific examples of the alkyl groups having 1 to 6 carbon atoms in which one or more hydrogen atoms have been substituted with groups having an amide bond include —(CH₂)ᵣNHC(═O) CH₃ and —(CH₂)ᵣC(═O) NH₂—. In the formula, r is an integer of 1 to 6.

The organic group having at least one double bond or triple bond is preferably any of an organic group having 6 to 12 carbon atoms and having an aromatic hydrocarbon, an organic group having 3 to 10 carbon atoms and having an aromatic heterocycle, an alkenyl group having 2 to 8 carbon atoms, and an alkynyl group having 3 to 8 carbon atoms. The organic group having at least one double bond or triple bond may be linear or branched.

Examples of the organic groups having 6 to 12 carbon atoms and having an aromatic hydrocarbon include, for example, a phenyl group, a methoxyphenyl group, a dimethoxyphenyl group, a cyanophenyl group, a dicyanophenyl group, a fluorinated phenyl group, a naphthyl group, a methoxynaphthyl group, a benzyl group, a methoxybenzyl group, a phenethyl group, a methoxyphenethyl group, a fluorinated phenethyl group, a naphthylmethyl group, and a naphthylethyl group. When the aromatic hydrocarbon has a substituent, a position to which the substituent is bonded may be anywhere.

Examples of the organic groups having 3 to 10 carbon atoms and having an aromatic heterocycle include a pyrrolyl group, a pyrazolyl group, a methylpyrazolyl methyl group, an imidazolyl group, a furyl group, a furfuryl group, an oxazolyl group, an isoxazolyl group, a thienyl group, a (thienyl)methyl group, a (thienyl)ethyl group, a thiazolyl group, a methylthiazolyl ethyl group, an isothiazolyl group, a pyridyl group, a pyrimidinyl group, a pyridazinyl group, a pyrazinyl group, an indolinyl group, a benzofuranyl group, a benzothienyl group, a benzoimidazolyl group, a benzoxazolyl group, a benzothiazolyl group, a benzopyrazolyl group, a benzoisoxazolyl group, a benzoisothiazolyl group, a quinolyl group, an isoquinolyl group, a quinazolinyl group, a quinoxalinyl group, a phthalazinyl group, and a cinnolinyl group.

Examples of the alkenyl groups having 2 to 8 carbon atoms include, for example, a vinyl group, an allyl group, a 1-propenyl group, an isopropenyl group, a 3-butenyl group and its structural isomers, a 4-pentenyl group and its structural isomers, a 5-hexenyl group and its structural isomers, a 6-heptenyl group and its structural isomers, and a 7-octenyl group and its structural isomers.

Examples of the alkynyl groups having 3 to 8 carbon atoms include, for example, a 1-propynyl group, a propargyl group, a 3-butynyl group and its structural isomers, a 4-pentynyl group and its structural isomers, a 5-hexynyl group and its structural isomers, a 6-heptynyl group and its structural isomers, and a 7-octynyl group and its structural isomers.

From the viewpoint of availability and/or facilitation of synthesis, R⁷ in formula (1-6) is preferably one group selected from the group consisting of a hydrogen atom, a methyl group, an ethyl group, an n-propyl group, an isopropyl group, a 2,2,2-trifluoroethyl group, a 2,2,3,3,3-pentafluoropropyl group, a 2,2,2,2,2,2-hexafluoroisopropyl group, a 2-cyanoethyl group, a 3-cyanopropyl group, a 4-cyanobutyl group, a phenyl group, a methoxyphenyl group, a cyanophenyl group, a phenethyl group, a thienylethyl group, an N-methylpyrazolylmethyl group, an allyl group, a 3-butenyl group, a 4-pentenyl group, a propargyl group, a 3-butynyl group, and a 4-pentynyl group. Among these, more preferred is one group selected from the group consisting of a hydrogen atom, a methyl group, an ethyl group, an n-propyl group, a 2,2,2-trifluoroethyl group, a 2,2,3,3,3-pentafluoropropyl group, a 3-cyanopropyl group, a 4-cyanobutyl group, a methoxyphenyl group, a cyanophenyl group, an allyl group, and a 3-butenyl group.

### End Groups Represented by Formulae (1-7) and (1-8)

In formula (1-7), i represents an integer of 1 to 6. i is preferably an integer of 1 to 4 and more preferably 1 or 2 because i facilitates a proportion of a fluorine atom in a fluorine-containing ether compound molecule to be ensured. R⁸ and R⁹ are represented by formula (1-9). R⁸ and R⁹ are the same or different.

In formula (1-8), j represents an integer of 0 to 6. j is preferably an integer of 0 to 4 and more preferably 0 or 2 because j facilitates a proportion of a fluorine atom in a fluorine-containing ether compound molecule to be ensured. k and L each independently represent an integer of 1 to 6. k and L are preferably integers of 1 to 4 and more preferably 1 or 2 because they facilitate a proportion of a fluorine atom in a fluorine-containing ether compound molecule to be ensured. k and L are the same or different. k and L are preferably the same because a fluorine-containing ether compound is easily produced. R¹⁰ and R¹¹ are hydrogen atoms or represented by formula (1-9). R¹⁰ and R¹¹ are the same or different.

### Partial Structure Represented by Formula (1-9)

In formula (1-9), p represents an integer of 2 to 6. p is preferably an integer of 2 to 4 and more preferably 2 or 3 because p facilitates a proportion of a fluorine atom in a fluorine-containing ether compound molecule to be ensured. q represents 1 or 2. When q is 2, p in each [—(CH₂)ₚ—O-]are the same or different. q is preferably 1 because it facilitates a proportion of a fluorine atom in a fluorine-containing ether compound molecule to be ensured.

In the fluorine-containing ether compound of the present embodiment, R¹—R²— and —R⁵—R⁶ in formula (1) preferably have the same structure. In other words, the fluorine-containing ether compound represented by formula (1) preferably has a symmetric structure at both ends.

When n is 1, it is preferable that R¹—R²— and —R⁵—R⁶ have the same structure, furthermore, the PFPE chains represented by R³ and R^{3′} are the same. Also, when n is 2, it is preferable that R¹—R²— and —R⁵—R⁶ have the same structure, furthermore, the PFPE chains represented by R³ and two R^{3′} are the same, and the divalent linking groups represented by two R⁴s are the same. This is because such a fluorine-containing ether compound can be easily produced and its production cost is low. Note that, R³ being the same as one or two R^{3′}s also includes the case where a structure of a repeating unit of the PFPE chain is the same, but an average degree of polymerization is different.

The number-average molecular weight (Mn) of the fluorine-containing ether compound of the present embodiment is preferably in a range of 500 to 10,000, more preferably in the range of 700 to 7,000, and still more preferably in the range of 800 to 4,000.

When the number average molecular weight is 500 or more, a lubricant containing the fluorine-containing ether compound of the present embodiment is less likely to evaporate. Therefore, when the number average molecular weight is 500 or more, a fluorine-containing ether compound becomes capable of forming a lubricating layer which is less likely to cause pick-up and spin-off. Also, when the number-average molecular weight is 10,000 or less, a fluorine-containing ether compound having a not too high viscosity and suitable for a lubricant is obtained. The number-average molecular weight of the fluorine-containing ether compound is more preferably 4,000 or less, since it has a viscosity easy to be handled when applied to a lubricant.

The fluorine-containing ether compound of the present embodiment particularly preferably has the number-average molecular weight in a range of 1,000 to 3,000. A lubricating layer including the fluorine-containing ether compound having a number-average molecular weight in the range of 1,000 to 3,000 does not deteriorate in coverage even though its film thickness is thin, and has favorable resistance to chemical substances and wear resistance. The fluorine-containing ether compound having such a number-average molecular weight range has the most favorable balance of performance from the viewpoints of inhibiting pick-up and spin-off in a lubricating layer containing this compound and making the lubricating layer thin.

The phrase “a proportion of molecules with the same molecular weight contained in the fluorine-containing ether compound is 90% or more in terms of a peak area ratio based on high-performance liquid chromatography” can also be interpreted as meaning that a proportion of molecules represented by formula (1s) to 100% of the fluorine-containing ether compound represented by formula (1) is 90% or more in terms of a peak area ratio based on high-performance liquid chromatography.

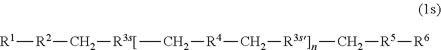

(wherein the same symbols as in formula (1) represent the same structures or values as in formula (1). R^{3s} and R^{3s′} are each independently the perfluoropolyether chain represented by formula (1f-1).).

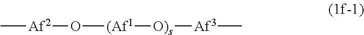

(wherein the same symbols as in formula (1f) represent the same structures as in formula (1f). s represents a degree of polymerization and is any one of integers of 1 to 13.).

In formula (1s), the same symbols as in formula (1) represent the same structures or values as in formula (1), and specific examples thereof are the same as those explained for formula (1). In formula (1f-1), the same symbols as in formula (1f) represent the same structures as in formula (1f), and specific examples thereof are the same as those described for formula (1f).

In formula (1f-1), s is any one of integers of 1 to 13. Therefore, the phrase “a proportion of molecules represented by formula (1s)” refers to a proportion of a molecule in which the number of repetitions (degree of polymerization) of the repeating units represented by (Af¹—O) in a perfluoropolyether chain is single.

The higher this proportion, the more molecules with single degrees of polymerization there are, and the fewer molecules with different degrees of polymerization there are. Such molecules can be prepared by making the number of repetitions (degree of polymerization) of the repeating units represented by (Af¹—O) in a raw material single. [Lubricant for magnetic recording media]

The lubricant for magnetic recording media of the present embodiment contains the fluorine-containing ether compound represented by the above formula (1).

The fluorine-containing ether compound represented by formula (1) has a perfluoropolyether chain represented by R³ and R^{3′}. The perfluoropolyether chain has a repeating unit represented by (Af¹—O) as shown in formula (1f). Therefore, even though the fluorine-containing ether compound represented by formula (1) has a single structure (in other words, even though structures of R¹, R², R³, R⁴, R^{3′}, R⁵, R⁶ and the value of n in formula (1) are the same), the molecular weight of a molecule contained in the fluorine-containing ether compound represented by formula (1) varies depending on the number of repetitions (degree of polymerization) of the repeating unit represented by (Af¹—O) in a perfluoropolyether chain.

This is because it is difficult to produce a compound in which the number of repetitions of the repeating unit represented by (Af¹—O) is completely single, by using, for example, raw materials of the fluorine-containing ether compound, the synthesis conditions, or a subsequent treatment method. However, it is difficult to adequately define a molecular weight variation using conventional characteristics such as a molecular weight distribution. For this reason, in the present invention, a fluorine-containing ether compound with less molecular weight variation is defined in terms of a peak area ratio based on high-performance liquid chromatography.

In the present embodiment, the fluorine-containing ether compound represented by formula (1) has a proportion of molecules with the same molecular weight of 90% or more in terms of a peak area ratio based on high-performance liquid chromatography, so that the number of repetitions (degree of polymerization) of the repeating unit represented by (Af¹—O) in a perfluoropolyether chain is almost uniform, and a variation in molecular weight is very small.

In other words, a proportion of molecules represented by formula (1s) to 100% of the fluorine-containing ether compound represented by formula (1) is 90% or more in terms of a peak area ratio based on high-performance liquid chromatography, so that the number of repetitions (degree of polymerization) of the repeating unit represented by (Af¹—O) in a perfluoropolyether chain is almost uniform, and the variation in molecular weight is very small.

The lubricant for magnetic recording media of the present embodiment is preferably substantially free of other materials used as lubricants (compounds other than the fluorine-containing ether compound represented by formula (1) and fluorine-containing ether compounds represented by formula (1) but having a structure different from that of the main component), and is more preferably composed of the fluorine-containing ether compound represented by formula (1).

The content of other materials in the lubricant is preferably 5% by mass or less, more preferably 3% by mass or less, and still more preferably 1% by mass or less. That is, in the lubricant for magnetic recording media of the present embodiment, the content of fluorine-containing ether compound represented by formula (1), which is as main component, is preferably 95% by mass or more and more preferably 97% by mass or more.

### [Method for Producing Lubricant for Magnetic Recording Media]

The method for producing a lubricant for magnetic recording media of the present embodiment preferably includes a step of synthesizing the fluorine-containing ether compound represented by formula (1) using the fluorinated polyalkylene glycol represented by the following formula (1f′) as a raw material. A proportion of molecules with the same molecular weight contained in the fluorinated polyalkylene glycol represented by formula (1f′) is 90% or more in terms of a peak area ratio based on high-performance liquid chromatography.

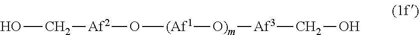

(wherein Af¹, Af², Af³ and m correspond to those in formula (1f). In formula (1), when n is 1 or 2 and when R³ and one or two R^{3′}s are not all the same, formula (1f′) represents a fluorinated polyalkylene glycol corresponding to each R³ and R^{3′}.)

m in formula (1f′) “corresponding to those in formula (1f)” means that m representing an average degree of polymerization in formula (1f′) is almost the same as m representing an average degree of polymerization in formula (1f), but the values are not necessarily identical. This is because there may be a difference in the average degree of polymerization before and after a step of synthesizing the fluorine-containing ether compound represented by formula (1). In the following formulae, average degrees of polymerization in two formulae “corresponding to each other”, mean that they are almost the same, but the values do not necessarily identical.

In the method for producing a lubricant for magnetic recording media of the present embodiment, using the fluorinated polyalkylene glycol represented by formula (1f′) in which a proportion of molecules with the same molecular weight is 90% or more as a raw material allows the fluorine-containing ether compound represented by formula (1) to be finally obtained also having a proportion of molecules with the same molecular weight of 90% or more.

Conventionally, in known lubricants, a proportion of molecules with the same molecular weight contained in a fluorinated polymer is at most approximately 15 to 20%. This is because a PFPE polymer used as a raw material has a wide molecular weight distribution. As methods for adjusting a molecular weight distribution of the fluorinated polymer include, for example, distillation, liquid-liquid separation extraction, column chromatography, and supercritical extraction. However, a difference in physical properties of the fluorinated polymer due to a difference in the number of repetitions is small, and it has been difficult to increase a proportion of molecules with the same molecular weight to 90% or more from the viewpoint of purification costs.

The fluorinated polyalkylene glycol represented by formula (1f′) is preferably synthesized by a method including the following steps (A) to (C):

The step (A) of liquid-phase fluorinating the both-end-protected polyalkylene glycol, represented by the following formula (2), in which a proportion of molecules with the same molecular weight is 90% or more in terms of a peak area ratio based on high-performance liquid chromatography;
the step (B) of reacting the obtained fluorinated reactant, represented by the following formula (3), with an alcohol having 1 to 6 carbon atoms; and
the step (C) of reducing the both-end-esterified product obtained represented by the following formula (4).

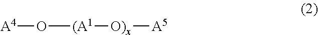

(wherein A¹ represents a divalent hydrocarbon group having a linear or branched structure having 2 to 5 carbon atoms. When A¹ is a linear structure, repeating units represented by (A¹—O) are all the same. When A¹ is a branched structure, a structure in which an orientation direction of A¹ is interchanged from left to right in the repeating unit represented by (A¹—O) is optionally partially included. x represents an average degree of polymerization and is a real number of 3 to 15. A⁴ and A⁵ each independently represent a protecting group for a hydroxy group.)

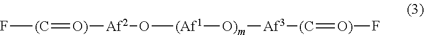

(wherein Af¹, Af², Af³ and m correspond to those in formula (1f).)

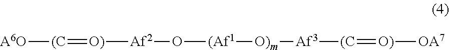

(wherein Af¹, Af², Af³ and m correspond to those in formula (1f). A⁶ and A⁷ each independently represent an alkyl group having 1 to 6 carbon atoms.)

### <Step Prior to Step (A)>

The both-end-protected polyalkylene glycol represented by formula (2) in which a proportion of molecules with the same molecular weight is 90% or more in terms of a peak area ratio based on high-performance liquid chromatography that is used in the step (A) is not particularly limited, but is preferably a compound synthesized by a polyether synthesis step described below.

The both-end-protected polyalkylene glycol represented by formula (2) may be obtained by protecting hydroxy groups at both ends of the polyalkylene glycol represented by formula (2′) in which a proportion of molecules with the same molecular weight is 90% or more in terms of a peak area ratio based on high-performance liquid chromatography with the protecting groups represented by A⁴ and A⁵ in formula (2). The polyalkylene glycol represented by formula (2′) is also preferably synthesized by the following polyether synthesis step.

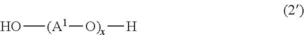

(wherein A¹ and x correspond to those in formula (2).)

### [Polyether Synthesis Step]

The method for producing a lubricant for magnetic recording media of the present embodiment preferably includes, prior to the step (A), a step (hereinafter may be referred to as a “polyether synthesis step”) of carrying out a nucleophilic substitution reaction of reacting two or more compounds having a polyether chain or a monomer unit constituting the polyether chain to synthesize a polyether compound having the repeating unit represented by (A¹—O). By such a synthesis step, a polyether compound (for example, the both-end-protected polyalkylene glycol represented by formula (2) or the polyalkylene glycol represented by formula (2′)) can be obtained in which a proportion of molecules with the same molecular weight is 90% or more, as a result of which the fluorine-containing ether compound represented by formula (1) in which a proportion of molecules with the same molecular weight is 90% or more can also be obtained.

In the polyether synthesis step, specifically, by carrying out reactions by any one of the following “reaction 1”, “reaction 2”, or “reaction 3”, or in combination of a plurality of the reactions selected from the “reaction 1”, “reaction 2”, or “reaction 3”, a polyether compound having a proportion of molecules with the same molecular weight of 90% or more in terms of a peak area ratio based on high-performance liquid chromatography, can be synthesized.

The number of times to carry out the reaction selected from the “reaction 1”, “reaction 2”, or “reaction 3” may be adjusted according to a structure of a polyether compound to be synthesized and a structure of a compound having a polyether chain (or a monomer unit constituting the polyether chain) used for the reaction. When carrying out the reactions in combination of a plurality of the reactions selected from the “reaction 1”, “reaction 2”, or “reaction 3”, the order of the reactions is not particularly limited.

### “Reaction 1”

In the reaction 1, a compound having a leaving group at one end of a polyether chain (or a monomer unit constituting the polyether chain) and having a protected hydroxy group at the other end (hereinafter may be referred to as a “compound having a leaving group at one end”) is reacted with a compound having a hydroxy group at one end of a polyether chain (or a monomer unit constituting the polyether chain) and a protected hydroxy group at the other end (hereinafter may be referred to as a “protected diol compound”) to elongate a polyether chain.

The compound having a leaving group at one end can be reacted with the protected diol compound at a molar ratio of approximately 1:1, to elongate the polyether chain at one end of the protected diol compound. Specifically, for example, by using the compound represented by Formula (G) as the compound having a leaving group at one end and by using the compound represented by Formula (H) as the protected diol compound, a polyether chain can be elongated to obtain the polyether compound represented by Formula (G+H).

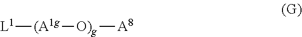

(The group —(A^{1g}—O)_{g}— is a moiety of the structure represented by —O—(A¹—O)ₓ— in formula (2). L¹ represents a leaving group. A⁸ represents a protecting group for a hydroxy group. g is an integer of 1 or greater.)

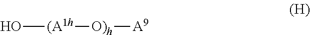

(The group —(A^{1h}-O)ₕ— is a moiety of the structure represented by —O—(A¹—O)ₓ— in formula (2). A⁹ represents a protecting group for a hydroxy group. h is an integer of 1 or greater.)

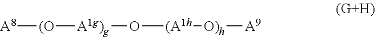

wherein the definitions of the symbols are the same as those of Formula (G) and Formula (H).

Leaving groups in the compound having a leaving group at one end for use can be, for example, a halogeno group, a p-toluenesulfonyloxy group (tosyloxy group), a methanesulfonyloxy (mesyloxy) group, a trifluoromethanesulfonyloxy (triflyloxy) group, a nonafluorobutanesulfonyloxy group, a fluorosulfonyloxy group, a chloromethanesulfonyloxy group, and a bromobenzenesulfonyloxy group.

As a protecting group for a hydroxy group in the protected diol compound, the protecting groups exemplified as A⁴ and A⁵ in formula (2) described later can be used.

The polyether compound obtained by the “reaction 1” has, at each end thereof, the protected hydroxy group derived from the end not involved in the reaction in the compound having a leaving group at one end, and the protected hydroxy group derived from the end not involved in the reaction in the protected diol compound. The polyether compound obtained by the “reaction 1” may be used as is as the both-end-protected polyalkylene glycol represented by formula (2), or a compound in which after one or both of the protected hydroxy groups have been deprotected, the hydroxy groups are protected with another protecting group may be used as the both-end-protected polyalkylene glycol represented by formula (2). Also, after one or both of the protected hydroxy groups in the polyether compound obtained by the “reaction 1” are deprotected, the “reaction 1”, “reaction 2”, or “reaction 3”, may be subsequently carried out to further elongate a polyether chain.

### “Reaction 2”

In the reaction 2, the compound having a leaving group at one end of a polyether chain (or a monomer unit constituting the polyether chain) and having a protected hydroxy group at the other end (a compound having a leaving group at one end) is reacted with a compound having hydroxy groups at both ends of a polyether chain (or a monomer unit constituting the polyether chain) (hereinafter may be referred to as a “diol compound”) to elongate a polyether chain.

The compound having a leaving group at one end can be reacted with the diol compound at a molar ratio of approximately 2:1 to elongate the polyether chain at both ends of the diol compound. Specifically, for example, by using the compound represented by Formula (G) described above as the compound having a leaving group at one end and by using the compound represented by Formula (I) as the diol compound, a polyether chain can be elongated to obtain the polyether compound represented by Formula (2G+I).

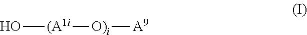

(The group —(A¹ⁱ-O)ᵢ— is a moiety of the structure represented by —O—(A¹—O)ₓ— in formula (2). i is an integer of 1 or greater.)

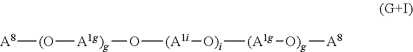

wherein the definitions of the symbols are the same as those of Formula (G) and Formula (I).

The polyether compound obtained by the “reaction 2” has, at both ends thereof, the protected hydroxy groups derived from the ends not involved in the reaction, in the compounds having a leaving group at one end. The polyether compound obtained by the “reaction 2” may be used as it is as the both-end-protected polyalkylene glycol represented by formula (2), or a compound in which after one or both of the protected hydroxy groups have been deprotected, the hydroxy groups are protected with another protecting group may be used as the both-end-protected polyalkylene glycol represented by formula (2). Also, after one or both of the protected hydroxy groups in the polyether compound obtained by the “reaction 2” are deprotected, the “reaction 1”, “reaction 2”, or “reaction 3”, may be subsequently carried out to further elongate a polyether chain.

### “Reaction 3”

In the reaction 3, the compound having a hydroxy group at one end of a polyether chain (or a monomer unit constituting the polyether chain) and having a protected hydroxy group at the other end (a protected diol) is reacted with a compound having leaving groups at both ends of a polyether chain (or a monomer unit constituting the polyether chain) (hereinafter may be referred to as a “compound having leaving groups at both ends”) to elongate a polyether chain.

The protected diol compound can be reacted with the compound having leaving groups at both ends at a molar ratio of approximately 2:1, to elongate the polyether chain at both ends of the compound having leaving groups at both ends. Specifically, for example, by using the compound represented by Formula (H) described above as the protected diol compound and by using the compound represented by Formula (J) as the compound having leaving groups at both ends, a polyether chain can be elongated to obtain the polyether compound represented by Formulae (2H+J).

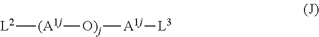

(The group —(A^{1j}-O)-A^{1j}— is a moiety of the structure represented by —O—(A¹—O)ₓ— in formula (2). L² and L³ each independently represent a leaving group. j is an integer of 0 or greater.)

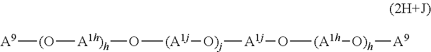

wherein the definitions of symbols are the same as those in Formula (H) and Formula (J).

As the leaving group in the compound having leaving groups at both ends, leaving groups exemplified as the leaving group in the compound having a leaving group at one end can be used.

The polyether compound obtained by the “reaction 3” has, at both ends thereof, the protected hydroxy groups derived from the ends not involved in the reaction in the protected diol compounds. The polyether compound obtained by the “reaction 3” may be used as it is as the both-end-protected polyalkylene glycol represented by formula (2), or a compound in which after one or both of the protected hydroxy groups have been deprotected, the hydroxy groups are protected with another protecting group may be used as the both-end-protected polyalkylene glycol represented by formula (2). Also, after one or both of the protected hydroxy groups in the polyether compound obtained by the “reaction 3” are deprotected, the “reaction 1”, “reaction 2”, or “reaction 3”, may be subsequently carried out to further elongate a polyether chain.

The polyether compound obtained by the polyether synthesis step may have hydroxy groups at both ends, or each hydroxy group at one end or both ends may be protected by a protecting group. The protecting group for the hydroxy group may be the same protecting group as that of the both-end-protected polyalkylene glycol represented by formula (2) or a different protecting group. When both ends of the polyether compound obtained by the polyether synthesis step are the same protecting groups as those of the both-end-protected polyalkylene glycol represented by formula (2), the polyether compound can be used as is in the step (A). When the both ends of the polyether compound are hydroxy groups or the hydroxy groups of the polyether compound are protected by a different protecting group from that of the both-end-protected polyalkylene glycol represented by formula (2), after carrying out a step of protecting the hydroxy group with the same protecting group as that of the both-end-protected polyalkylene glycol represented by formula (2), the polyether compound can be used in the step (A).

### <Step (A)>

In the method for producing a lubricant for magnetic recording media of the present embodiment, the step (A) is a step of liquid-phase fluorinating the both-end-protected polyalkylene glycol represented by formula (2) in which a proportion of molecules with the same molecular weight is 90% or more in terms of a peak area ratio based on high-performance liquid chromatography.

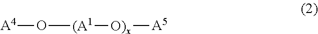

wherein A¹ represents a divalent hydrocarbon group having a linear or branched structure having 2 to 5 carbon atoms. A¹ in formula (2) may be selected according to a structure of Af¹ in the fluorinated polyalkylene glycol represented by formula (1f′) to be obtained. Specific examples of A¹ include, for example, —CH₂—CH₂—, —CH₂—CH₂—CH₂—, —CH₂—CH(CH₃)—, —CH(CH₃)—CH₂—, —CH₂—CH₂—CH₂—CH₂—, —CH(CH₃)—CH₂—CH₂—, —CH₂—CH(CH₃)—CH₂—, —CH₂—CH₂—CH(CH₃)—, —CH₂—CH₂—CH₂—CH₂—CH₂—, —CH(CH₃)—CH₂—CH₂—CH₂—, —CH₂—CH(CH₃)—CH₂—CH₂—, —CH₂—CH₂—CH(CH₃)—CH₂—, —CH₂—CH₂—CH₂—CH(CH₃)—, and —CH(CH₃)—CH₂—CH(CH₃)—.

In formula (2), when A¹ is a linear structure, A¹ in each repeating unit represented by (A¹—O) is the same, and when A¹ is a branched structure, a structure in which an orientation direction of A¹ in each repeating unit represented by (A¹—O) is interchanged from left to right is optionally partially included. For example, when A¹ is a branched structure, a bonding manner between each repeating unit represented by (A¹—O) is not limited to a head-to-tail bond, and may include a head-to-head bond and tail-to-tail bond.

A⁴ and A⁵ each independently represent a protecting group for a hydroxy group. Examples of the protecting group for the hydroxy group include an acyl group, an alkoxycarbonyl group, a silyl group, and an alkyl group optionally having a substituent. A⁴ and A⁵ are the same or different. When A⁴ and A⁵ are the same, synthesis is easy, which is preferable.

The acyl group is preferably represented by —(C═O)—R³ wherein R¹³ is a hydrogen atom or a hydrocarbon group having 1 to 8 carbon atoms which may have a substituent. The hydrocarbon group more preferably has 1 to 3 carbon atoms.

When R¹³ is an alkyl group having 1 to 8 carbon atoms, which may have a substituent, the alkyl group may be linear or branched. Examples of the substituent include, for example, an alkoxy group, a fluoro group, a chloro group, and a bromo group.

When R¹³ is an aryl group having 1 to 8 carbon atoms, which may have a substituent, the substituent may be an alkoxy group, a fluoro group, a chloro group, a bromo group, an acetoxy group, a nitro group, and the like.

Specific examples of the acyl group include a formyl group, an acetyl group, an ethoxyacetyl group, a fluoroacetyl group, a difluoroacetyl group, a trifluoroacetyl group, a chloroacetyl group, a dichloroacetyl group, a trichloroacetyl group, a bromoacetyl group, a dibromoacetyl group, a tribromoacetyl group, a propionyl group, a 2-chloropropionyl group, a 3-chloropropionyl group, a pentafluoropropionyl group, a butyryl group, a 2-chlorobutyryl group, a 3-chlorobutyryl group, a 4-chlorobutyryl group, a 2-methylbutyryl group, a 2-ethylbutyryl group, a heptafluorobutyryl group, a valeryl group, a 2-methylvaleryl group, a 4-methylvaleryl group, a perfluorovaleryl group, a hexanoyl group, a perfluorohexanoyl group, a heptanoyl group, a perfluoroheptanoyl group, an octanoyl group, a perfluorooctanoyl group, a nonanoyl group, a perfluorononanoyl group, an isobutyryl group, an isovaleryl group, a pivaloyl group, a benzoyl group, an o-chlorobenzoyl group, a m-chlorobenzoyl group, a p-chlorobenzoyl group, an o-acetoxy benzoyl group, a m-acetoxy benzoyl group, a p-acetoxy benzoyl group, an o-methoxybenzoyl group, a m-methoxybenzoyl group, a p-methoxybenzoyl group, an o-nitrobenzoyl group, a m-nitrobenzoyl group, a p-nitrobenzoyl group, an o-fluorobenzoyl group, a m-fluorobenzoyl group, a p-fluorobenzoyl group, and a pentafluorobenzoyl group.

Specific examples of the alkoxycarbonyl group include a methoxycarbonyl group, an ethoxycarbonyl group, a 2,2,2-trichloroethoxycarbonyl group, and an allyloxycarbonyl group.

Specific examples of the silyl group include a trimethylsilyl group, a triethylsilyl group, a t-butyldimethylsilyl group, and a t-butyldiphenylsilyl group.

Examples of the alkyl group optionally having a substituent include an alkyl group with a substituent selected from the group consisting of an alkoxy group, an aryl group, and a halogenated acyl group, and an alkyl group without a substituent. The number of carbon atoms of the alkyl group is not particularly limited, and an alkyl group having 1 to 8 carbon atoms is usually used.

Specific examples of the alkyl group having an alkoxy group include a methoxymethyl group, a methoxyethoxymethyl group, and a 1-ethoxyethyl group. The alkyl group having an alkoxy group may be a cyclic ether that forms an acetal structure or a ketal structure together with an oxygen atom derived from the hydroxy group in formula (2), and specific examples thereof include, for example, a 2-tetrahydropyranyl group.

Specific examples of the alkyl group having an aryl group include a benzyl group, a trityl group, an o-methoxybenzyl group, a m-methoxybenzyl group, and a p-methoxybenzyl group.

Specific examples of the alkyl group having a halogenated acyl group include, for example, —CH₂C(═O)F, —CH₂C(═O)Cl, —CH₂CH₂C(═O) F, —CH₂CH₂C(═O)Cl, —CH₂CH₂CH₂C(═O) F, and —CH₂CH₂CH₂C(═O)Cl.

Specific examples of the alkyl group without a substituent include a methyl group, an ethyl group, an n-propyl group, an i-propyl group, an n-butyl group, and a t-butyl group.

Among the above-described groups, A⁴ and A⁵ are more preferably acyl groups, further preferably acetyl groups, trifluoroacetyl groups, propionyl groups, pentafluoropropionyl groups, butyryl groups, and heptafluorobutyryl groups, and particularly preferably acetyl groups or trifluoroacetyl groups.

The both-end-protected polyalkylene glycol represented by the above formula (2) (hereinafter may be referred to as a “raw material compound”) is used to introduce an inert gas, a fluorine gas, and a solvent into a reactor and then to carry out liquid-phase fluorination.

The equivalent of fluorine gas introduced into a reactor is preferably 1.0 to 5.0 equivalents, and more preferably 1.1 to 3.0 equivalents relative to the number of moles of hydrogen atoms contained in a raw material compound. The equivalent of fluorine gas being 1.0 equivalent or more relative to the number of moles of hydrogen atoms contained in a raw material compound facilitates sufficient proceeding of fluorination reaction. The equivalent of fluorine gas being 5.0 equivalents or less relative to the number of moles of hydrogen atoms contained in a raw material compound, can prevent an unconsumed fluorine gas from being wasted.

A concentration of a fluorine gas to be distributed in a reactor is preferably 1 to 30% by volume, more preferably 10 to 20% by volume, based on the total volume of distribution gas (a fluorine gas+an inert gas). The fluorine gas concentration of 1% by volume or more can prevent the reaction time from becoming long due to a reduced reaction rate. The fluorine gas concentration being 30% by volume or less can prevent reaction runaway and generation of side reaction. Pressure in a reactor upon introduction of fluorine gas is preferably 0.08 to 0.12 MPa, more preferably normal pressure (0.1 MPa) to 0.115 MPa. The pressure of 0.12 MPa or lower can prevent the reaction runaway and generation of side reaction.

An inert gas is distributed into a reactor so that the fluorine gas concentration falls within the above range. The inert gas and fluorine gas may be introduced in separate systems, or mixture gas of inert gas and fluorine gas, in which the fluorine gas has been preliminarily diluted with the inert gas, may be introduced into a reactor. As the inert gas, preferred are, for example, a nitrogen gas, a helium gas, and an argon gas because of their availability and handleability.

A solvent used in the fluorination reaction is not particularly limited, but is preferably a solvent that has high solubility for raw material compounds and a fluorinated reactant that is a product, and is more preferably a solvent that does not react with the raw material compounds, the product, and the fluorine gas. Specifically, preferred is a solvent that is fully halogen-substituted and free of carbon-carbon unsaturated bonds. The solvent that was fully halogen-substituted and free of carbon-carbon unsaturated bonds is free of C—H bonds or carbon-carbon unsaturated bonds, so that the C—H bond or carbon-carbon unsaturated bond in the solvent does not react with a fluorine gas, making it possible to prevent the amount of fluorine gas used from increasing and prevent temperature due to reaction heat from rising. A decomposition reaction of the raw material compound by hydrogen fluoride, which is generated in the case of reaction between the C—H bond and the fluorine gas, also does not occur, which is preferred.

Examples of the solvents used in the fluorination reaction include, for example, a perhalogenated alkane, a perhalogenated polyether, a perhalogenated carboxylic acid or anhydride thereof. The solvent may be used singly or in combinations with two or more types.

The perhalogenated alkane having 2 to 8 carbon atoms is preferred. The perhalogenated alkane containing a fluorine atom and a chlorine atom is more preferable from the viewpoint of solubility for a raw material compound, and includes, for example, dichlorotetrafluoroethane, trichlorotrifluoroethane, dichlorohexafluoropropane, and tetrachlorohexafluorobutane.

Examples of commercially available perhalogenated polyethers include, for example, DEMNUM (registered trademark) by DAIKIN INDUSTRIES, LTD., FLUORINERT (registered trademark) by 3M Japan Limited, GALDEN (registered trademark) by Solvay Specialty Polymers Japan K.K., KRYTOX (registered trademark) by The Chemours Company.

Examples of the perhalogenated carboxylic acid or anhydride thereof include, for example, trifluoroacetic acid and trifluoroacetic anhydride.

The solvent used in the fluorination reaction is preferably introduced into a reactor before introducing a raw material compound. It is also preferable that before introducing the raw material compound, an inert gas and fluorine gas are distributed in the reactor to saturate the solvent with the fluorine gas.

A method for introducing the raw material compound is preferably a method for preparing a raw material solution obtained by dissolving the raw material compound in a solvent and supplying the raw material solution into the reactor while distributing an inert gas and fluorine gas through the reactor. As the solvent dissolving the raw material compound, solvents exemplified for the fluorination reaction can be used and is preferably the same as the solvent used in the fluorination reaction.

A concentration of the raw material compound in the reactor may be adjusted according to its solubility in the solvent, and it is preferably 0 to 3.0 mol/L and more preferably 0 to 1.5 mol/L. A supply rate of the raw material solution to the reactor may be adjusted according to a concentration and flow rate of the fluorine gas being distributed so that the equivalent of the fluorine gas relative to the raw material compound falls within the above range. Note, however, it is also possible to carry out the fluorination reaction in gas phase without using a solvent. It is noted that in the gas phase reaction, cleavage of C—C single bonds may occur during the fluorination reaction, resulting in production of many types of by-products.

In the step (A), a temperature inside the reactor upon introduction of fluorine gas is preferably −30 to 60° C. and more preferably −20 to 30° C.

In one embodiment, a temperature in a reactor upon the fluorine gas introduction is preferably 20 to 60° C. and more preferably 20 to 30° C. The temperature in the reactor is preferably equal to or higher than the boiling point of hydrogen fluoride (20° C.) in order to efficiently remove hydrogen fluoride that was produced as a by-product. In the case of the temperature being 20° C. or higher, hydrogen fluoride does not remain and a decomposition reaction of a raw material is less likely to occur, which is preferred. In the case of the temperature being 60° C. or lower, reaction runaway and generation of side reaction can be prevented, which is preferred.

In another embodiment, a temperature in a reactor upon fluorine gas introduction may be −30 to 20° C. or −20 to 0° C. In this case, for the purpose of efficiently removing the by-product of hydrogen fluoride, it is preferable to increase a dilution ratio of a nitrogen gas to the fluorine gas or to use a hydrogen fluoride scavenger. Examples of the hydrogen fluoride scavengers include alkali metal fluorides such as sodium fluoride and potassium fluoride, and an organic base such as a trialkylamine.

As a reactor in the fluorination reaction, a pressure-resistant reactor is preferably used, and an autoclave is usually used. A material of the reactor is not particularly limited, however, preferred is a metal vessel made of stainless steel or nickel, or a vessel coated with a fluororesin since it is less likely to react with the fluorine gas.

In the fluorination reaction, both of a distribution type reactor and a batch type reactor can be employed.

In the case of the distribution type reactor, a flow rate of a raw material solution supplied to the reactor is not particularly limited, but is adjusted according to the equivalent of fluorine gas relative to the amount of raw material compound, a size of the reactor, and pressure in the reactor, for example. The flow rate of the raw material solution supplied to the reactor is preferably 0.5 to 100 mmol/min and more preferably 2 to 30 mmol/min, based on the number of moles of hydrogen atoms contained in the raw material compound.

In the case of the batch type reactor, a pressure-controlled fluorine gas may be introduced from an entrance of the reactor as much as is consumed in the reaction.

In the step (A), the compound represented by formula (2f) is produced in which a hydrogen atom bonded to a carbon atom contained in the both-end-protected polyalkylene glycol represented by formula (2) is substituted with a fluorine atom.

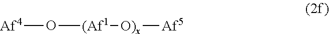

(Af¹ in formula (2f) is the same as that in formula (1f). Af⁴ represents a group in which all hydrogen atoms of A⁴ in formula (2) were substituted with fluorine atoms. Af⁵ represents a group in which all hydrogen atoms of A⁵ in formula (2) were substituted with fluorine atoms. x corresponds to that of formula (2).)

Af¹ in formula (2f) corresponds to a structure in which all hydrogen atoms of A¹ in formula (2) were substituted with fluorine atoms, and is the same as Af¹ in formula (1f).

The compound represented by formula (2f) is quickly converted to the fluorinated reactant represented by the following formula (3) in a reaction system.

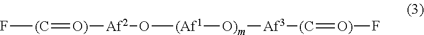

wherein Af¹, Af², Af³ and m correspond to those in formula (1f).

In formula (3), the structure represented by —O—(Af¹—O)ₘ-corresponds to the structure represented by —O—(Af¹—O)ₓ— in formula (2f) excluding the structural units arranged at both ends. Therefore, m in formula (3) is 2 less than the number of x in formulae (2) and (2f).

In formula (3), the structures represented by —(C═O)—Af²— and —Af³—(C═O)— are structures derived from the structural units arranged at each end of the structure represented by —O—(Af¹—O)ₓ— in formula (2f). Therefore, the structures of Af² and Af³ in formula (3) are determined according to a structure of a structural unit arranged at the end in formula (2f), and are the same perfluorohydrocarbon groups as those of Af² and Af³ in formula (1f). The number of carbon atoms contained in Af² and Af³ is 1 less than the number of carbon atoms contained in the structural unit arranged at the end in formula (2f).

### [Optional Step]

In the production method of the present embodiment, after the step (A) and before the step (B), a step of introducing a perhalogenated unsaturated hydrocarbon compound into a reactor while distributing an inert gas and fluorine gas through the reactor may be optionally carried out.

In a later stage of the fluorination reaction in the step (A), a reaction rate of the fluorination reaction may be lowered. For this reason, it is preferable that after the step (A), the step of introducing the perhalogenated unsaturated hydrocarbon compound into the reactor while distributing an inert gas and fluorine gas through the reactor. Introducing the perhalogenated unsaturated hydrocarbon compound allows an unsaturated bond in the perhalogenated unsaturated hydrocarbon compound to react with the fluorine gas to generate fluorine radicals. Since generated fluorine radicals react with a raw material compound to allow for proceeding of fluorination, this step carried out can promote the fluorination reaction.

As an inert gas used in the step of introducing the perhalogenated unsaturated hydrocarbon compound, those exemplified in the step (A) can be used. Flow rates of the inert gas and fluorine gas are preferably adjusted so that a concentration of the fluorine gas distributed through a reactor is within the range exemplified in the step (A).

Examples of the perhalogenated unsaturated hydrocarbon compounds include, for example, hexafluorobenzene, hexachlorobenzene, chloropentafluorobenzene, trichlorotrifluorobenzene, decafluorobiphenyl, octafluoronaphthalene, tetrachloroethylene, trichlorofluoroethylene, dichlorodifluoroethylene, trichlorotrifluoropropene, and dichlorotetrafluoropropene, and of these, hexafluorobenzene that is available and handleable, is particularly preferable.

By using the perhalogenated unsaturated hydrocarbon compound, as is the case of using an unsaturated hydrocarbon compound having a C—H bond such as benzene, the fluorine gas is not consumed in fluorination of a C—H bond in the unsaturated hydrocarbon compound, and the amount of fluorine gas used does not increase, which is preferable.

As a method for introducing the perhalogenated unsaturated hydrocarbon compound, it is preferable to dissolve the perhalogenated unsaturated hydrocarbon compound in a solvent and introduce and distribute a certain amount thereof into a reactor. The amount of perhalogenated unsaturated hydrocarbon compound distributed is preferably 1/50 to 1/5 mol per mol, more preferably 1/30 to 1/10 mol per mol in terms of the number of moles of unsaturated bonds in the perhalogenated unsaturated hydrocarbon compound, relative to the amount of fluorine gas distributed. When the amount of perhalogenated unsaturated hydrocarbon compound distributed is 1/50 mol per mol or more, proceeding of the fluorination reaction is not delayed, thereby making it possible to prevent a reaction time from prolonging. When the amount of perhalogenated unsaturated hydrocarbon compound distributed is 1/5 mol per mol or less, the reaction runaway and generation of side reaction can be prevented.

Pressure in a reactor upon introduction of the perhalogenated unsaturated hydrocarbon compound is preferably 0.08 to 0.12 MPa, more preferably normal pressure (0.1 MPa) to 0.115 MPa. A temperature in the reactor upon introduction of the perhalogenated unsaturated hydrocarbon compound is preferably −30 to 60° C. and more preferably −20 to 30° C.

A solvent used in the step of introducing the perhalogenated unsaturated hydrocarbon compound is preferably the same solvent as that in the step (A). In the case of using a solvent different from that used in the step (A), those exemplified in the step (A) can be used.

In a case in which the perhalogenated unsaturated hydrocarbon compound is dissolved in a solvent and supplied to a reactor as a solution, a concentration of the perhalogenated unsaturated hydrocarbon compound in the supplying solution can be adjusted according to its solubility in the solvent, and the concentration is preferably 0.01 to 100 mol/L and more preferably 0.1 to 10 mol/L based on the number of moles of unsaturated bonds in the perhalogenated unsaturated hydrocarbon compound.

### <Step (B)>

In the method for producing a lubricant for magnetic recording media of the present embodiment, the step (B) is a step of reacting the fluorinated reactant represented by formula (3) obtained in the step (A) with an alcohol having 1 to 6 carbon atoms.

The fluorinated reactant produced in the step (A) may easily react with moisture in the air to be converted to a carboxylic acid compound. For this reason, the step (B) is preferably carried out in consideration of handleability in the subsequent step. Particularly, when A⁴ and A⁵ in the raw material compound represented by formula (2) are acyl groups, the step (B) is preferably carried out.

Examples of the alcohols having 1 to 6 carbon atoms include, for example, methanol, ethanol, propanol, butanol, pentanol, and hexanol. Among them, methanol is preferred.

A reaction temperature in the step (B) is preferably −30 to 60° C. and more preferably −20 to 30° C. Reaction pressure is preferably 0.08 to 0.12 MPa and more preferably normal pressure (0.1 MPa) to 0.115 MPa.

The amount of alcohol introduced is preferably 2 to 10 equivalents and more preferably 3 to 5 equivalents, relative to the number of moles of reactive ends contained in the fluorinated reactant created in the step (A) (theoretical amount based on the number of moles of a raw material compound).

In the raw material compound represented by formula (2), when A¹ is —CH₂—CH₂— and A⁴ and A⁵ are acetyl groups, reactions in the steps (A) to (B) are shown in formula (1). The following formula denotes a reaction when methanol is used in the step (B).

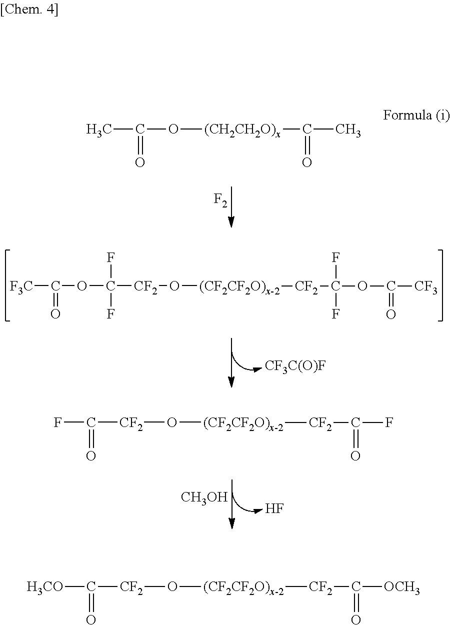

The step (B) carried out yields the both-end esterified product represented by the following formula (4) from the fluorinated reactant represented by formula (3).

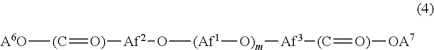

(wherein Af¹, Af², Af³ and m correspond to those in formula (1f). A⁶ and A⁷ each independently represent an alkyl group having 1 to 6 carbon atoms.)

In formula (4), the structures represented by A⁶O— and —OA⁷ are derived from alcohols used in the step (B).

### [Optional Step]

A product of the step (A) or step (B) can be isolated as a residue obtained by having distilled off a solvent. Before the solvent is distilled off, it is preferable to wash the product with alkaline water in order to remove a by-product such as hydrogen fluoride generated in the fluorination reaction. The alkaline water is not particularly limited, but sodium carbonate water or sodium hydrogen carbonate water is preferred due to their availability and handleability. After having washed with alkaline water followed by having collected a solvent layer from the separated two layers, in order to completely remove water and hydrogen fluoride, a hydrogen fluoride scavenger and a drying agent are preferably added and stirred.

Examples of the hydrogen fluoride scavengers include alkali metal fluorides such as sodium fluoride and potassium fluoride, and an organic base such as a trialkylamine. The hydrogen fluoride scavenger is preferably solid alkali metal fluoride because of easy separation thereof, and particularly preferably sodium fluoride.

As the drying agent, sodium sulfate or magnesium sulfate is preferable.

By filtering off the solid and then distilling off the solvent, the product of the step (A) or step (B) can be isolated as well as the collected solvent can be easily reused, thereby enabling reducing a loss of an expensive solvent such as a fully halogen-substituted compound.

### <Step (C)>

In the method for producing a lubricant for magnetic recording media of the present embodiment, the step (C) is a step of carrying out a reduction reaction of the both-end-esterified product represented by formula (4) obtained in the step (B).

In the step (C), a known method for reducing the ester can be employed. For example, a method can be employed whereby the both-end-esterified product obtained in the step (B) is mixed with a reducing agent in a solvent.

The solvent used in the step (C) is preferably an alcohol having 1 to 5 carbon atoms. Since the both-end-esterified product obtained in the step (B) has high solubility, as the alcohol, ethanol is preferably used.

The reducing agent used in the step (C) is preferably at least one selected from the group consisting of alkali metal salts of borohydride compounds, such as sodium borohydride and lithium borohydride; alkaline earth metal salts of borohydride compounds, such as magnesium borohydride and calcium borohydride; and aluminum hydride salts, such as lithium aluminum hydride and sodium aluminum hydride. Among these, sodium borohydride is particularly preferred due to its availability and handleability.

By carrying out the step (C), the fluorinated polyalkylene glycol represented by formula (1f′) can be produced.

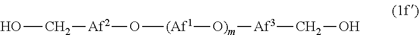

(wherein Af¹, Af², Af³ and m correspond to those in formula (1f). In formula (1), when n is 1 or 2 and when R³ and one or two R^{3′}s are not all the same, formula (1f′) represents a fluorinated polyalkylene glycol corresponding to each R³ and R^{3′}.)

In the step (A) of the method for producing a lubricant for magnetic recording media of the present embodiment, the both-end-protected polyalkylene glycol represented by formula (2) in which a proportion of molecules with the same molecular weight is 90% or more in terms of a peak area ratio based on high-performance liquid chromatography is used as a raw material compound. For this reason, the fluorinated polyalkylene glycol represented by formula (1f′) obtained through the steps (A) to (C) also has a proportion of molecules with the same molecular weight of 90% or more in terms of a peak area ratio based on high-performance liquid chromatography.

When synthesizing the fluorine-containing ether compound represented by formula (1) using the fluorinated polyalkylene glycol represented by formula (1f′) in which a proportion of molecules with the same molecular weight is 90% or more in terms of a peak area ratio based on high-performance liquid chromatography as a raw material, a conventionally known method can be employed. When n in formula (1) is 0, for example, the method described in Patent Literature 1 can be employed. When n in formula (1) is 1, for example, the method described in Patent Literature 2 can be employed. When n in formula (1) is 2, for example, the method described in Patent Literature 3 can be employed.

In formula (1), when n is 1 or 2 and when R³ and one or two R^{3′}s are not all the same, formula (1f′) represents a fluorinated polyalkylene glycol corresponding to each of R³ and R^{3′}. When n is 1 or 2 in formula (1) and R³ and R^{3′} are all the same, one type of fluorinated polyalkylene glycol represented by formula (1f′) may be used as a raw material, facilitating synthesis of the fluorine-containing ether compound represented by formula (1), which is preferable.

As described above, the phrase “a proportion of molecules with the same molecular weight contained in the fluorine-containing ether compound is 90% or more in terms of a peak area ratio based on high-performance liquid chromatography” can also be said to mean that a proportion of molecules represented by formula (1s) to 100% of the fluorine-containing ether compound represented by formula (1) is 90% or more in terms of a peak area ratio based on high-performance liquid chromatography.”

Therefore, in the method for producing a lubricant for magnetic recording media of the present embodiment, the phrase “a proportion of molecules with the same molecular weight contained in the fluorinated polyalkylene glycol represented by formula (1f′) is 90% or more in terms of a peak area ratio based on high-performance liquid chromatography” can also be said to mean that a proportion of molecules represented by formula (1f′-1) to 100% of the fluorinated polyalkylene glycol represented by formula (1f′) is 90% or more in terms of a peak area ratio based on high-performance liquid chromatography.

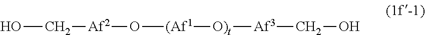

(wherein the same symbols as in formula (1f′) represent the same structures as in formula (1f′). t represents a degree of polymerization and is any one of integers of 1 to 13.)

In formula (1f′-1), the same symbols as in formula (1f′) represent the same structures as in formula (1f′), and specific examples thereof are the same as those explained for formula (1f′). t in formula (1f′-1) represents any one of integers of 1 to 13. Therefore, the phrase “a proportion of molecules represented by formula (1f′-1)” refers to a proportion of molecules in which the number of repetitions (degree of polymerization) of the repeating unit represented by (Af¹—O) in a perfluoropolyether chain is single.

In the step (A) of the method for producing a lubricant for magnetic recording media of the present embodiment, the phrase “the both-end-protected polyalkylene glycol represented by formula (2) in which a proportion of molecules with the same molecular weight is 90% or more in terms of a peak area ratio based on high-performance liquid chromatography” can also be said to refer to the both-end-protected polyalkylene glycol represented by formula (2) in which a proportion of molecules represented by formula (2-1) is 90% or more in terms of a peak area ratio based on high-performance liquid chromatography.

A step of liquid-phase fluorinating the both-end-protected polyalkylene glycol represented by formula (2) in which a proportion of molecules represented by formula (2-1) is 90% or more in terms of a peak area ratio based on high-performance liquid chromatography, can also be said to refer to the step (A-1). A step of reacting the obtained fluorinated reactant represented by formula (3) with an alcohol having 1 to 6 carbon atoms can also be said to refer to the step (B-1), a step of carrying out the reduction reaction of the obtained both-end esterified product represented by formula (4) can be further said to refer to the step (C-1), and these can also be combined to be employed in the method for producing a lubricant for magnetic recording media of the present embodiment.

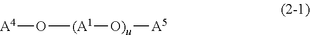

(wherein the same symbols as in formula (2) represent the same structures as in formula (2). u represents a degree of polymerization and is any one of integers of 3 to 15.)

In formula (2-1), the same symbols as in formula (2) represent the same structures as in formula (2), and specific examples thereof are the same as those described for formula (2). u in formula (2-1) is any one of integers of 3 to 15.

Therefore, the phrase “a proportion of molecules represented by formula (2-1)” refers to a proportion of molecules in which the number of repetitions (degree of polymerization) of the repeating unit represented by (A¹—O) is single.

Also, in the step prior to the step (A) of the method for producing a lubricant for magnetic recording media of the present embodiment, the phrase “the polyalkylene glycol represented by formula (2′) in which a proportion of molecules with the same molecular weight is 90% or more in terms of a peak area ratio based on high-performance liquid chromatography” can also be said to refer to the polyalkylene glycol represented by formula (2′) in which a proportion of molecules represented by formula (2′-1) is 90% or more in terms of a peak area ratio based on high-performance liquid

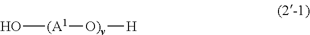

(wherein the same symbols as in formula (2′) represent the same structures as in formula (2′). v represents a degree of polymerization and is any one of integers of 3 to 15.)

In formula (2′-1), the same symbols as in formula (2′) represent the same structures as in formula (2′), and specific examples thereof are the same as those described for formula (2′). v in formula (2′-1) is any one of integers of 3 to 15.

Therefore, the phrase “a proportion of molecules represented by formula (2′-1)” refers to a proportion of molecules in which the number of repetitions (degree of polymerization) of the repeating unit represented by (A¹—O) is single. “Method for measuring a proportion of molecules with the same molecular weight”

In the fluorine-containing ether compound represented by formula (1), the fluorinated polyalkylene glycol represented by formula (1f′), the both-end-protected polyalkylene glycol represented by formula (2), or the polyalkylene glycol represented by formula (2′), each proportion of molecules with the same molecular weight contained therein is a value calculated from each peak area ratio based on high-performance liquid chromatography, and is determined by the method described in Examples.

As HPLC conditions, appropriate conditions are used according to a structure of a compound to be measured. For example, when an object to be measured is the fluorinated polyalkylene glycol represented by formula (1f′), the both-end-protected polyalkylene glycol represented by formula (2), or the polyalkylene glycol represented by formula (2′), the “HPLC conditions (i)” described in Examples is preferably used. When an object to be measured is the fluorine-containing ether compound represented by formula (1), on the other hand, the “HPLC conditions (ii)” described in Examples is preferably used. The “HPLC conditions (i)” is a condition of using a refractive index detector (RID) as a detector, and the “HPLC conditions (ii)” is a condition of using an evaporative light scattering detector (ELSD) as a detector.

When an object to be measured is the fluorinated polyalkylene glycol represented by formula (1f′), the both-end-protected polyalkylene glycol represented by formula (2), or the polyalkylene glycol represented by formula (2′), a low molecular weight component may not be detected by an evaporative light scattering detector (ELSD), or its peak intensity may be extremely weak.

When an object to be measured is the fluorine-containing ether compound represented by formula (1), a content percentage of the fluorine-containing ether compound with different numbers of repetitions may not be accurately measured by a differential refractive index detector (RID). This is because a PFPE chain moiety of the fluorine-containing ether compound and other non-fluorinated moieties act against each other to cancel it out, resulting in rendering an extremely small difference of the refractive indices with respect to the refractive index of a mobile phase or being unable to detect a peak. [Magnetic recording medium]The magnetic recording medium of the present embodiment includes at least a magnetic layer, a protective layer, and a lubricating layer sequentially provided on a substrate.

In the magnetic recording medium of the present embodiment, one or two or more underlayers can be provided between the substrate and the magnetic layer, if necessary. Also, an adhesive layer and/or a soft magnetic layer can be provided between the underlayer and the substrate.

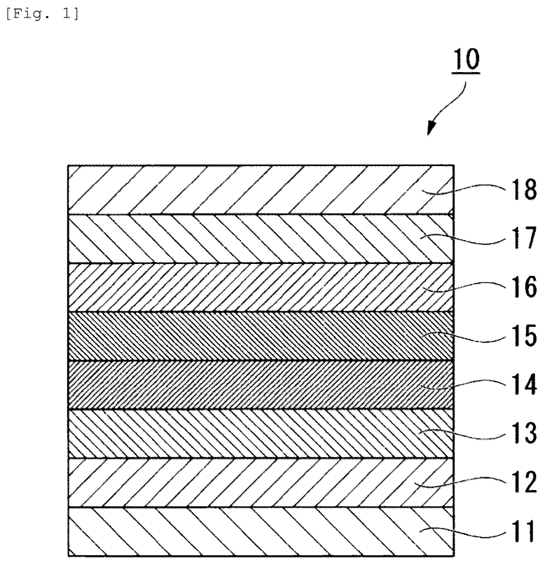

FIG. 1 is a schematic cross-sectional view illustrating one embodiment of the magnetic recording medium of the present invention.

The magnetic recording medium 10 of the present embodiment has a structure in which an adhesive layer 12, a soft magnetic layer 13, a first underlayer 14, a second underlayer 15, a magnetic layer 16, a protective layer 17, and a lubricating layer 18 are sequentially provided on a substrate 11.

### “Substrate”

The substrate 11 for use can be, for example, a non-magnetic substrate in which a film made of NiP or a NiP alloy was formed on a base material made of a metal or an alloy material such as A¹ or an A¹ alloy.

The substrate 11 for use may also be a non-magnetic substrate made of a non-metallic material such as glass, ceramics, silicon, silicon carbide, carbon, or a resin, or a non-magnetic substrate in which a film of NiP or a NiP alloy was formed on a base material made of such a non-metallic material.

The glass substrate is rigid and has excellent smoothness, which is thereby suitable for achieving a high recording density. An example of the glass substrate includes, for example, an aluminosilicate glass substrate. The glass substrate which is a chemically strengthened aluminosilicate glass substrate is particularly suitable.

A main surface of the substrate 11 preferably has an ultra-smooth surface roughness with Rmax of 6 nm or less and Ra of 0.6 nm or less. The surface roughness Rmax and Ra used herein is based on the provisions of JIS B0601.

### “Adhesive Layer”

The adhesive layer 12 prevents corrosion of the substrate 11 from proceeding, which occurs when the substrate 11 and the soft magnetic layer 13 provided on the adhesion layer 12 were arranged in contact with each other.

A material of the adhesive layer 12 can be appropriately selected from, for example, Cr, an Cr alloy, Ti, an Ti alloy, CrTi, NiAl, or an AlRu alloy. The adhesive layer 12 can be formed, for example, by a sputtering method.

### “Soft Magnetic Layer”

The soft magnetic layer 13 preferably has a structure stacked with a first soft magnetic film, an intermediate layer made of a Ru film, and a second soft magnetic film in this order. That is, the soft magnetic layer 13 preferably has a structure in which the intermediate layer made of a Ru film is sandwiched between the two soft magnetic films to allow the soft magnetic films above and below the intermediate layer to be bonded in an anti-ferro-coupling (AFC) manner.

Examples of materials of the first soft magnetic film and the second soft magnetic film include, for example, a CoZrTa alloy and a CoFe alloy.

The CoFe alloy used for the first soft magnetic film and second soft magnetic film is preferably added with any one of Zr, Ta, and Nb. This promotes amorphization of the first soft magnetic film and second soft magnetic film. As a result, it becomes possible to improve an orientation of the first underlayer (seed layer) and reduce a floating height of a magnetic head.

The soft magnetic layer 13 can be formed, for example, by a sputtering method.

### “First Underlayer”

The first underlayer 14 is a layer for controlling orientations and crystal sizes of the second underlayer 15 and the magnetic layer 16 to be provided on the first underlayer 14.

Examples of the first underlayer 14 include underlayers made of, for example, a Cr layer, a Ta layer, a Ru layer, or a CrMo alloy layer, a CoW alloy layer, a CrW alloy layer, a CrV alloy layer, and a CrTi alloy layer.

The first underlayer 14 can be formed, for example, by a sputtering method.

### “Second Underlayer”

The second underlayer 15 is a layer that controls an orientation of the magnetic layer 16 to be favorable. The second underlayer 15 is preferably a layer made of Ru or a Ru alloy.

The second underlayer 15 may be a layer made of one layer, or may be made of a plurality of layers. When the second underlayer 15 is made of a plurality of layers, all the layers may be composed of the same material, or at least one layer may be composed of a different material.

The second underlayer 15 can be formed, for example, by a sputtering method.

“Magnetic Layer” The magnetic layer 16 is made of a magnetic film, an axis of easy magnetization of which is oriented perpendicular or horizontal to a substrate surface. The magnetic layer 16 is a layer containing Co and Pt. In order to improve a SNR (signal to noise ratio) characteristics, the magnetic layer 16 may be a layer containing, for example, an oxide, Cr, B, Cu, Ta, or Zr.

Examples of the oxides contained in the magnetic layer 16 include, for example, SiO₂, SiO, Cr₂O3, CoO, Ta₂O₃, and TiO₂.

The magnetic layer 16 may be composed of one layer, or may be composed of a plurality of magnetic layers made of a material with different composition.

For example, when the magnetic layer 16 is composed of three layers of a first magnetic layer, a second magnetic layer, and a third magnetic layer, which have been stacked in this order from the bottom, the first magnetic layer preferably has a granular structure formed of a material containing Co, Cr, and Pt and further containing an oxide.

As the oxide contained in the first magnetic layer, for example, oxides of, for example, Cr, Si, Ta, Al, Ti, Mg, and Co are preferably used. Among these, for example, TiO₂, Cr₂O₃, and SiO₂ can be particularly preferably used. Also, the first magnetic layer is preferably made of a composite oxide to which two or more types of the oxides are added. Among these, for example, Cr₂O₃—SiO₂, Cr₂O₃—TiO₂, and SiO₂—TiO₂ can be particularly preferably used.

The first magnetic layer can contain one or more elements selected from B, Ta, Mo, Cu, Nd, W, Nb, Sm, Tb, Ru, or Re, in addition to Co, Cr, Pt, and the oxide.

For the second magnetic layer, the same material as that of the first magnetic layer can be used. The second magnetic layer preferably has a granular structure.

The third magnetic layer preferably has a non-granular structure formed of a material containing Co, Cr, and Pt and not containing an oxide. The third magnetic layer can contain one or more elements selected from B, Ta, Mo, Cu, Nd, W, Nb, Sm, Tb, Ru, Re, or Mn, in addition to Co, Cr, and Pt.

When the magnetic layer 16 is formed of a plurality of magnetic layers, it is preferable to arrange a non-magnetic layer between the adjacent magnetic layers. When the magnetic layer 16 is made of three layers of the first magnetic layer, the second magnetic layer, and the third magnetic layer, it is preferable to arrange a non-magnetic layer between the first magnetic layer and the second magnetic layer and between the second magnetic layer and the third magnetic layer.

The non-magnetic layer provided between the adjacent magnetic layers of the magnetic layer 16 can suitably use, for example, Ru, a Ru alloy, a CoCr alloy, or a CoCrX1 alloy (Xl represents one or two or more elements selected from Pt, Ta, Zr, Re, Ru, Cu, Nb, Ni, Mn, Ge, Si, 0, N, W, Mo, Ti, V, and B).

For the non-magnetic layer provided between the adjacent magnetic layers of the magnetic layer 16, an alloy material containing an oxide, a metal nitride, or a metal carbide, is preferably used. Specifically, for example, SiO₂, A¹₂O₃, Ta₂O₅, Cr₂O₃, MgO, Y₂O₃, or TiO₂ can be used as the oxide. As the metal nitride, for example, AlN, Si₃N₄, TaN, or CrN can be used. As the metal carbide, for example, TaC, BC, or SiC can be used.

The non-magnetic layer can be formed by, for example, a sputtering method.

In order to achieve a higher recording density, the magnetic layer 16 is preferably a magnetic layer for perpendicular magnetic recording, an axis of easy magnetization of which is oriented perpendicular to a substrate surface. The magnetic layer 16 may be a magnetic layer for in-plane magnetic recording.

The magnetic layer 16 may be formed by any conventionally known method, such as a vapor deposition method and sputtering methods such as an ion beam sputtering method and a magnetron sputtering method. In general, the magnetic layer 16 is preferably formed by a sputtering method.

### “Protective Layer””

The protective layer 17 protects the magnetic layer 16. The protective layer 17 may be composed of one layer or a plurality of layers. Examples of materials for the protective layer 17 include, for example, carbon, carbon containing nitrogen and silicon carbide.

The protective layer 17 for use can preferably be a carbon-based protective layer and particularly an amorphous carbon protective layer. The protective layer 17 which is the carbon-based protective layer further enhances an interaction with a polar group (particularly a hydroxy group) contained in a fluorine-containing ether compound in the lubricating layer 18, which is preferred.

Adherence between the carbon-based protective layer and the lubricating layer 18 can be controlled by using the carbon-based protective layer of hydrogenated carbon and/or nitrogenated carbon and adjusting the hydrogen content and/or nitrogen content in the carbon-based protective layer. The hydrogen content in the carbon-based protective layer is preferably 3 atomic % to 20 atomic % when measured by hydrogen forward scattering (HFS). The nitrogen content in the carbon-based protective layer is preferably 4 atomic % to 15 atomic % when measured by X-ray photoelectron spectroscopy (XPS).

Hydrogen and/or nitrogen contained in the carbon-based protective layer is not required to be uniformly contained throughout the carbon-based protective layer. The carbon-based protective layer is preferably a compositionally gradient layer in which, for example, the protective layer 17 on the lubricating layer 18 side contains nitrogen and the protective layer 17 on the magnetic layer 16 side contains hydrogen. In this case, the adherence of the magnetic layer 16 and the lubricating layer 18 with the carbon-based protective layer is further improved. This is because the nitrogen in the protective layer 17 acts as an active site, promoting bonding with the lubricating layer 18. The hydrogen or nitrogen in the carbon-based protective layer acts as an active site.

A thickness of the protective layer 17 is preferably 1 nm to 7 nm. When the thickness of the protective layer 17 is 1 nm or more, sufficient performance as the protective layer 17 can be obtained. When the thickness of the protective layer 17 is 7 nm or less, it is preferable from the viewpoint of making the protective layer 17 thin.

As a deposition method of the protective layer 17, for example, a sputtering method using a target material containing carbon, a CVD (chemical vapor deposition) method using a hydrocarbon raw material such as ethylene or toluene, and an IBD (ion beam deposition) method can be used.

When the carbon-based protective layer is formed as the protective layer 17, for example, it can be formed by a DC magnetron sputtering method. In particular, when the carbon-based protective layer is formed as the protective layer 17, an amorphous carbon protective layer is preferably deposited by a plasma CVD method. The amorphous carbon protective layer formed by the plasma CVD method has a uniform surface with small roughness.

### “Lubricant Layer”

The lubricating layer 18 prevents the magnetic recording medium 10 from being contaminated. The lubricating layer 18 also reduces frictional force of a magnetic head of a magnetic recording and reproduction apparatus, which slides on the magnetic recording medium 10, and improves durability of the magnetic recording medium 10.

As shown in FIG. 1, the lubricating layer 18 is formed on and in contact with the protective layer 17. The lubricating layer 18 includes the above-described lubricant for magnetic recording media.

When the protective layer 17 arranged under the lubricating layer 18 is a carbon-based protective layer, the lubricating layer 18 is bonded particularly to the protective layer 17 with high bonding strength. As a result, the lubricating layer 18 having an even thin thickness facilitates the magnetic recording medium 10 in which a surface of the protective layer 17 is covered with a high coverage to be obtained, thereby enabling effectively preventing a surface of the magnetic recording medium 10 from being contaminated.

An average thickness of the lubricating layer 18 is preferably 0.5 nm (5 Å) to 2.0 nm (20 Å) and more preferably 0.5 nm (5 Å) to 1.0 nm (10 Å). When the average thickness of the lubricating layer 18 is 0.5 nm or more, the lubricating layer 18 with a uniform thickness is formed without being formed into an island or mesh shape. As a result, the lubricating layer 18 can cover a surface of the protective layer 17 with a high coverage. The lubricating layer 18 with an average thickness of 2.0 nm or less makes it possible for the lubricating layer 18 to be sufficiently thin and to render a sufficiently small floating height of the magnetic head.

When a surface of the protective layer 17 is not covered with the lubricating layer 18 with a sufficiently high coverage, an environmental substance adsorbed on a surface of the magnetic recording medium 10 passes through gaps in the lubricating layer 18 and intrudes under the lubricating layer 18. The environmental substance that have intruded under the lubricating layer 18 is adsorbed and bonded to the protective layer 17 to create pollutants. Then, upon magnetic recording and reproduction, these pollutants (aggregated components) adhere (transfer) to a magnetic head as smears, resulting in damage of the magnetic head or deterioration of magnetic recording and reproduction characteristics of a magnetic recording and reproduction apparatus.

Examples of the environmental substances that create pollutants include, for example, siloxane compounds (a cyclic siloxane and linear siloxane), ionic impurities, a hydrocarbon with a relatively high molecular weight such as octacosane, and a plasticizer such as dioctyl phthalate. Examples of metal ions contained in the ionic impurities can include, for example, sodium ions and potassium ions. Examples of inorganic ions contained in the ionic impurities can include, for example, chloride ions, bromide ions, nitrate ions, sulfate ions, and ammonium ions. Examples of organic ions contained in the ionic impurities can include, for example, oxalate ions and formate ions.

### “Method for Forming Lubricating Layer”

An example of a method for forming the lubricating layer 18 includes, for example, a method for preparing a magnetic recording medium in the course of production, in which each layer up to the protective layer 17 has been formed on the substrate 11, coating a surface of the protective layer 17 with a lubricating layer forming solution, and drying it.

The lubricant layer forming solution can be obtained, for example, by dispersing and dissolving the lubricant for magnetic recording media of the above embodiment in a solvent, if necessary, and adjusting its viscosity and concentration to be suitable for a coating method.

Examples of the solvents used in the lubricating layer forming solution include, for example, fluorine-based solvents such as Vertrel (registered trademark) XF (product name, manufactured by Chemours-Mitsui Fluoroproducts Co., Ltd.) and ASAHIKULIN (registered trademark) AE-3000 (product name, manufactured by AGC Inc.).

Examples of a coating method for the lubricating layer forming solution include, for example, a spin coating method, a spraying method, a paper coating method, and a dipping method, with the dipping method being preferred.

When using the dipping method, for example, the following method can be employed. First, the substrate 11 on which each layer up to the protective layer 17 has been formed is immersed in the lubricating layer forming solution placed in an immersion tank of a dip coater. Then, the substrate 11 is pulled up from the immersion tank at a predetermined speed. In this manner, a surface of the protective layer 17 of the substrate 11 is coated with the lubricating layer forming solution.

Using the dipping method allows the surface of the protective layer 17 to be uniformly coated with the lubricating layer forming solution, and the lubricating layer 18 can be formed on the protective layer 17 with a uniform thickness.

Using the lubricating layer forming solution containing the lubricant for magnetic recording media of the above-described embodiment enables, even though the number of substrates coated in a coating step increases, preventing the phenomenon whereby a proportion of fluorine-containing ether compounds with a small molecular weight in the lubricating layer forming solution is significantly reduced. Therefore, even though the number of substrates coated increases, wear resistance and resistance to chemical substances of the lubricating layer of a magnetic recording medium can be prevented from deteriorating.

Also, in order to prevent the wear resistance and resistance to chemical substances of the lubricating layer from deteriorating as the number of substrates coated increases, the need to frequently exchange the lubricating layer forming solution in the immersion tank can be reduced. This makes it possible to reduce a frequency of exchanging the lubricating layer forming solution and to enhance productivity of a coating step.

In the present embodiment, the substrate 11 on which the lubricating layer 18 has been formed is preferably subjected to heat treatment. The substrate 11 subjected to the heat treatment improves adhesiveness between the lubricating layer 18 and the protective layer 17 and improves adherence between the lubricating layer 18 and the protective layer 17.

A heat treatment temperature is preferably 100° C. to 180° C. When the heat treatment temperature is 100° C. or higher, an effect of improving adhesiveness between the lubricating layer 18 and the protective layer 17 is sufficiently obtained. Also, by adjusting the heat treatment temperature to 180° C. or lower, it is possible to prevent thermal decomposition of the lubricating layer 18 due to the heat treatment. A heat treatment time is preferably 10 minutes to 120 minutes.

In the present embodiment, in order to further improve adherence of the lubricating layer 18 to the protective layer 17, the lubricating layer 18 of the substrate 11 before or after the heat treatment may undergo treatment to irradiate with ultraviolet (UV) rays.

### Examples

The present invention will be described in more detail below with reference to Examples and Comparative Examples. It is noted that the present invention is not limited to the following Examples.

### [Nuclear Magnetic Resonance (M4R) Measurement Method]

Structures of each compound synthesized in the following Examples and Comparative Examples were identified from the results of ¹H-NMR and ¹⁹F-NMR measurements using a NMR apparatus (AVANCEIII-400) manufactured by Bruker GmbH.

Each measurement sample for NMR was prepared by dissolving approximately 10 mg of each compound in approximately 0.5 mL of deuterated chloroform (containing tetramethylsilane (TMS) as a reference substance) or deuterated acetone (containing TMS and hexafluorobenzene as reference substances). For compounds before carrying out the liquid-phase fluorination step, NMR measurements were made using deuterated chloroform, and for all compounds after the liquid-phase fluorination step, deuterated acetone was used. A reference for ¹H-NMR chemical shifts relative to tetramethylsilane was set to 0.0 ppm, and a reference for ¹⁹F-NMR chemical shifts relative to hexafluorobenzene was set to −164.7 ppm.

The number-average molecular weight (Mn) and an average degree of polymerization of each compound were calculated from the integrated intensity of each peak obtained by ¹H-NMR measurement or ¹⁹F-NMR measurement and the number of hydrogen atoms or fluorine atoms assigned to each peak in the following manner. When a plurality of values of average degrees of polymerization could be calculated from the identical NMR spectrum, an average value thereof was adopted as the value of the average degree of polymerization of the compound.

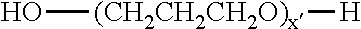

(x′ represents an average degree of polymerization.)

¹H-NMR (CDCl₃): 5 [ppm]=3.7-3.9 (4H), 3.4-3.7 (4 (x′-1)H), 2.0-3.0 (2H), 1.7-1.9 (2x¹H)

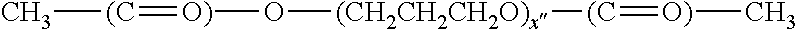

(x″ represents an average degree of polymerization.)

¹H-NMR (CDCl₃): 5 [ppm]=4.0-4.3 (4H), 3.4-3.6 (4 (x”-1)H), 2.0-2.1 (6H), 1.7-2.0 (2x″H)

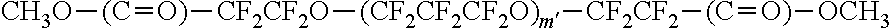

(m′ represents an average degree of polymerization.)

¹H-NMR (acetone-D₆) δ [ppm]=4.0-4.1 (6H)

¹⁹F-NMR (acetone-D₆): 5 [ppm]=−84.2-−84.5 (4m′F), −86.4--86.6 (4F), −122.5-−122.7 (4F), −130.0-−130.2 (2m′F)

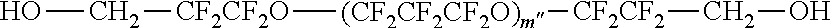

(m″ represents an average degree of polymerization.)

′H-NMR (acetone-D₆) δ [ppm]=5.1-5.2 (2H), 4.0-4.2 (4H)

¹⁹F-NMR (acetone-D₆) δ [ppm]=−84.2-−84.8 (4m″F), −86.4--87.3 (4F), −126.3-−126.7 (4F), −129.8-−130.2 (2m″F)

### [High-Performance Liquid Chromatography (HPLC) Measurement Method]

HPLC measurements of each compound synthesized in the following Examples and Comparative Examples were made under either HPLC conditions (i) or (ii) below. The content percentage of a constituent component for each number of repetitions was determined from a peak area ratio with respect to an elapsed time based on an area percentage method.

HPLC conditions (i): Gel permeation chromatography (GPC) Apparatus name: an HPLC Prominence UFLC manufactured by Shimadzu Corporation
Column: Shodex (registered trademark) KF-402HQ (1 column) and KF-401HQ (3 columns) connected in series, manufactured by Resonac Corporation
Column oven: 30° C.
Detector: Refractive index detector (RID)

### Eluent: Tetrahydrofuran

Flow rate: 0.2 mL/min
Injection volume: 1 μL
Sample preparation method: Approximately 50 mg of sample was dissolved in approximately 1 mL of tetrahydrofuran.
Standard material: “EasiVial PEG” manufactured by Agilent Technologies Inc.

Molecular weight distribution data were obtained by creating a calibration curve using standard materials.

HPLC conditions (ii): Normal phase chromatography Apparatus name: HPLC Prominence manufactured by Shimadzu Corporation
Column: Inertsil SIL-100A (length 75 mm x inner diameter 2.1 mm, silica particle size 5 μm, and pore size 10 nm) manufactured by GL Sciences Inc.
Column oven: 40° C.
Detector: Evaporative light scattering detector (ELSD) Eluent: ASAHIKLIN (registered trademark) AE-3000 manufactured by AGC Inc./2-propanol in a gradient manner
Total flow rate: 0.5 mL/min
Injection volume: 10 μL
Sample preparation method: Approximately 5 mg of sample was dissolved in approximately 1.5 mL of AE-3000.

Note that the values of Mw/Mn (Mw represents a weight-average molecular weight and Mn represents a number-average molecular weight) in Examples and Comparative Examples were determined from the above GPC measurement results. Also, when calculating a peak area ratio for peaks that could not be completely separated, based on the area percentage method, the peaks were vertically divided at the local minimum value or inflection point thereof to determine the corresponding peak areas.

### Example 1

### <Polyalkylene Glycol Synthesis Step>

A 1, 3-propanediol pentamer (HO—(CH₂CH₂CH₂O)₅—H) was synthesized by the following method.

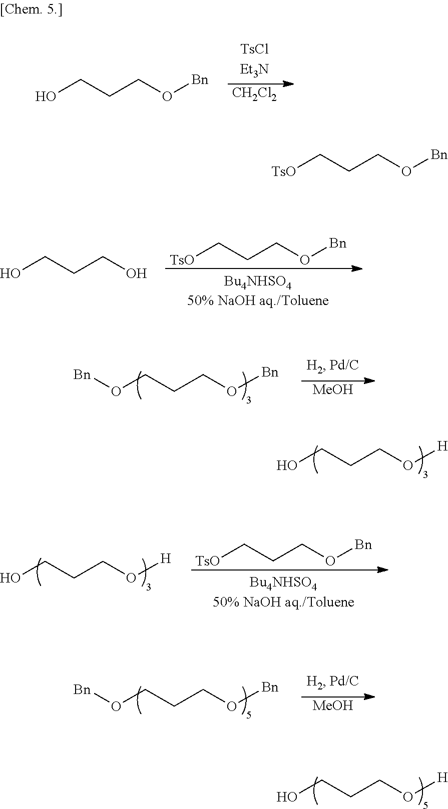

First, 3-(benzyloxy) propyl p-toluenesulfonate was synthesized by the following procedure.

A 3L three-neck flask with a thermometer, a dropping funnel and a stirrer was added with 3-benzyloxy-1-propanol (molecular weight 166.22, 149.60 g, 900 mmol, and 1.00 eq.) and dichloromethane (900 m L) and was cooled in an ice bath. p-Toluenesulfonyl chloride (molecular weight 190.64, 188.73 g, 990 mmol, and 1.10 eq.) was added in batch, and triethylamine (molecular weight 101.19, 109.29 g, 1080 mmol, and 1.20 eq.) was then added dropwise over 30 minutes. Thereafter, the ice bath was removed and the mixture was stirred at room temperature for 36 hours. The flask was cooled in an ice bath and was added with distilled water (1,000 mL). The two layers were separated, and the aqueous layer was extracted twice with dichloromethane (500 mL). The organic layers were combined and dried with sodium sulfate. After filtration of a drying agent, the filtrate was concentrated under reduced pressure to give 298 g of a crude product as yellow oil. The crude product was purified by silica gel column chromatography to yield 3-(benzyloxy)propyl p-toluenesulfonate (molecular weight 320.41, 253.76 g, 792 mmol, and yield 88%) as pale yellow oil.

Next, a 1, 3-propanediol trimer HO—(CH₂CH₂CH₂O) 3-H) was synthesized by the following procedure.

A 3L three-necked flask with a thermometer, a reflux tube, and a mechanical stirrer was added with 1,3-propanediol (molecular weight 76.10, 25.11 g, 330 mmol, and 1.00 eq.), 3-(benzyloxy)propyl p-toluenesulfonate (molecular weight 320.41, 253.76 g, 792 mmol, and 2.40 eq.), and toluene (1560 mL) and the mixture was stirred. Tetrabutylammonium hydrogen sulfate (molecular weight 339.54, 112.05 g, 330 mmol, and 1.00 eq.) and a 50% sodium hydroxide aqueous solution (1,305 g, 16.31 mol, and 49.43 eq.) were added and then heated to reflux with vigorous stirring. After 24 hours of heating, the reaction was stopped by pouring the reaction solution into 5% hydrochloric acid (6 L) while cooling the solution on ice. The reaction solution was transferred to a separating funnel to separate into two layers, and the aqueous layer was extracted three times with ethyl acetate (2 L). The organic layers were combined and dried with sodium sulfate. After filtration of a drying agent, the filtrate was concentrated under reduced pressure to give 98.5 g of a crude product as yellow oil. The crude product was purified by silica gel column chromatography to yield 1,3-propanediol trimer dibenzyl protected product (molecular weight 372.51, 87.28 g, 234 mmol, and yield 71%) as pale yellow oil.

A 3L three-necked flask was added with the synthesized 1,3-propanediol trimer dibenzyl protected product (molecular weight 372.51, 87.28 g, 234 mmol, and 1.00 eq.) and methanol (1,500 mL), and the mixture was stirred at room temperature to prepare a solution. 10% Pd/C (8.73 g) was added, the inside of the flask was substituted with hydrogen gas, and the solution was then vigorously stirred for 1.5 hours at room temperature. The reaction solution was filtered through Celite and the residue was washed with methanol (200 mL). The filtrate was concentrated and vacuum dried to give 87.6 g of a crude product as colorless oil. The crude product was purified by silica gel column chromatography to yield 1,3-propanediol trimer (molecular weight 192.26, 41.83 g, 218 mmol, and yield 93%) as colorless oil.

Subsequently, the 1,3-propanediol trimer HO—(CH₂CH₂CH₂O)₃—H) synthesized by the above procedure and 3-(benzyloxy)propyl p-toluenesulfonate were used as raw materials to repeat the same procedure as the synthesis of the 1,3-propanediol trimer and to obtain a 1,3-propanediol pentamer (molecular weight 308.42, 42.87 g, and 139 mmol). A ¹H-NMR measurement of the obtained 1,3-propanediol pentamer HO—(CH₂CH₂CH₂O)ₙₐ—H (na represents an average degree of polymerization) was made, and the average degree of polymerization na was found to be na=5.01. FIG. 2 shows the ¹H-NMR spectrum of the 1,3-propanediol pentamer. A proportion of molecules with the same molecular weight contained in the 1,3-propanediol pentamer was determined by a method under the HPLC conditions (i). As a result, a component (HO—(CH₂CH₂CH₂O)₅—H) with a degree of polymerization of 5 had the largest peak area ratio, with an area ratio of 97.93%.

### <Step of Introducing Protective Group to Hydroxy Groups at Both Ends>

The resulting 1,3-propanediol pentamer was reacted with acetyl chloride to acetylate hydroxy groups at both ends and to synthesize the compound represented by CH₃—(C═O)—O—(CH₂CH₂CH₂O)₅—(C═O)—CH₃.

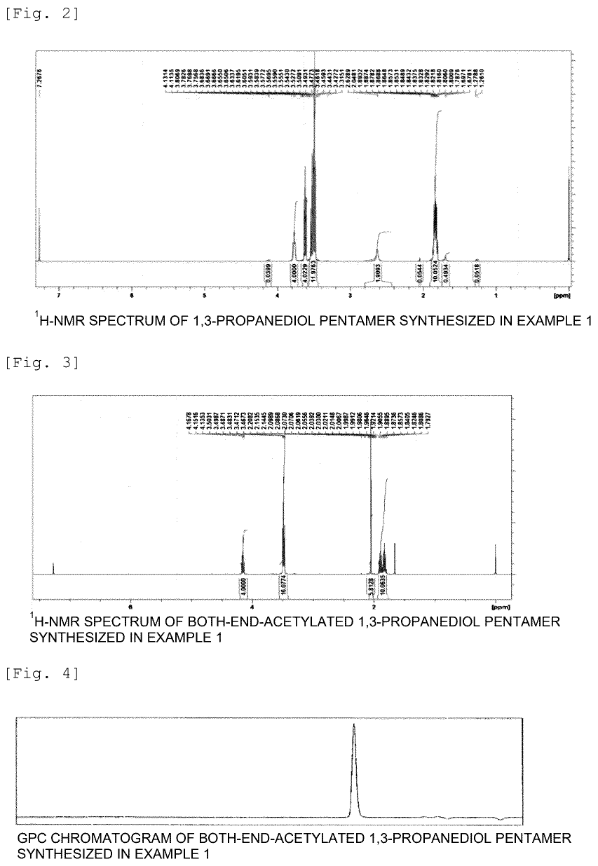

In such a manner, CH₃—(C═O)—O—(CH₂CH₂CH₂O)_{nb}—(C═O)—CH₃ (nb represents an average degree of polymerization. nb=5.03, Mn 394, and Mw/Mn=1.00) was obtained as a raw material compound for the next step. FIG. 3 shows the ¹H-NMR spectrum of the both-end acetylated 1,3-propanediol pentamer. A proportion of the component (CH₃—(C═O)—O—(CH₂CH₂CH₂O)₅—(C═O)—CH₃) with the degree of polymerization of 5 determined by the method under the HPLC conditions (i) was 99.75%. FIG. 4 shows the GPC chromatogram measured by the method under the HPLC conditions (i).

### <Liquid-Phase Fluorination Step (A)>

A 5 L autoclave was introduced with 3,100 mL of HFTCB (tetrachlorohexafluorobutane) and sealed. The autoclave was introduced with a nitrogen gas until the internal pressure reached 0.3 MPa, and an operation of slowly releasing the internal pressure to normal pressure was repeated 10 times to purge the autoclave. 15 g of CH₃—(C═O)—O—(CH₂CH₂CH₂O)_{nb}—(C═O)—CH₃ (nb represents an average degree of polymerization. nb=5.03, Mn 394, and Mw/Mn=1.00) obtained in the previous step was dissolved in 5.7 mL of HFTCB to prepare a raw material solution. A fluorine gas and a nitrogen gas were distributed at 588 mL/min and 4,600 mL/min, respectively, and while cooled to an internal temperature of 25° C., the raw material solution was introduced and distributed at a flow rate of 0.22 g/min on a solution mass basis, to carry out a fluorination reaction.

After the raw material solution was introduced and distributed, 1.87 g of C₆F₆ (hexafluorobenzene) was dissolved in 73 mL of HFTCB to prepare a C₆F₆ solution. A temperature inside the autoclave was adjusted to 25 to 30° C., and while distributing a fluorine gas at 150 mL/min and a nitrogen gas at 1,350 mL/min, the C₆F₆ solution was introduced and distributed at a flow rate of 0.83 g/min on a solution mass basis, to carry out a final reaction.

<Step (B) of Reacting Fluorinated Reactant with Alcohol>

After completion of introduction of C₆F₆ in the step (A), the fluorine gas and nitrogen gas continue to be distributed for 10 minutes, and thereafter the fluorine gas stopped being distributed, and the nitrogen gas was distributed at 1,350 mL/min for 1 hour to purge an inside of the autoclave. While distributing the nitrogen gas, 24 g of methanol was introduced.

A reaction solution collected from the autoclave was washed with sodium carbonate water. After separation of the solution into two layers, an organic solvent layer was collected, dried with sodium sulfate and sodium fluoride, and then the solid was filtered off. The solvent was removed using an evaporator to obtain 31 g of product (rate of collection 97% (rate of collection relative to the raw material compounds of the fluorination reaction in the step (A))). The product obtained was analyzed by ¹⁹F-NMR and confirmed to be CH₃O—(C═O)—CF₂CF₂O—(CF₂CF₂CF₂O)_{nc}—CF₂CF₂—(C═O)—OCH₃ (nc represents an average degree of polymerization. nc=3.04 and Mn 839).

### <Step (C) of Obtaining Fluorinated Polyalkylene Glycol by Reduction Reaction>

A reduction reaction of CH₃O—(C═O)—CF₂CF₂O—(CF₂CF₂CF₂O)_{nc}—CF₂CF₂—(C═O)—OCH₃ obtained in the step (B) was carried out using the following method.

A eggplant flask was added with 119 g of ethanol, cooled to 0° C., and then charged with 1.82 g of sodium borohydride. While cooling the solution to 0° C., 31 g of CH₃O—(C═O)—CF₂CF₂O—(CF₂CF₂CF₂O)_{nc}—CF₂CF₂—(C═O)—OCH₃ (nc represents an average degree of polymerization. nc=3.04, and Mn 839) was added dropwise at 2.7 g/min, and the mixture was washed down with 10 g of ethanol. The reaction was allowed to continue for 4.5 hours while the eggplant flask was brought to room temperature.

After completion of the reaction, 12.6 mL of 4 M hydrochloric acid was added dropwise and a pH of the solution was confirmed to be 3, and then a sodium hydrogen carbonate aqueous solution (6.4 g of sodium hydrogen carbonate and 76 mL of water) was added, and the pH of the solution was confirmed to be 8. The ethanol was removed using an evaporator, and 80 mL of water was added, and then the aqueous layer was extracted three times with 80 mL of AE-3000. Organic layers were combined and dried over magnesium sulfate, and the solids were then filtered off. The filtrate was concentrated under reduced pressure using an evaporator, yielding 28.5 g of crude product (rate of collection 98.5%). The crude product was purified by silica gel column chromatography to obtain 23.5 g (30.6 mmol, 81% yield) of fluorinated 1,3-propanediol pentamer HO—CH₂—CF₂CF₂O—(CF₂CF₂CF₂O)_{nd}—CF₂CF₂—CH₂—OH (nd represents an average degree of polymerization. nd=2.96 and Mn 769) as colorless oil. FIG. 5 shows a ¹⁹F-NMR spectrum of the fluorinated 1,3-propanediol pentamer. A component with the degree of polymerization of 3 (HO—CH₂—CF₂CF₂O—(CF₂CF₂CF₂O)₃—CF₂CF₂—CH₂—OH) determined by the method under the HPLC conditions (i) was a component having the largest peak area ratio, with an area ratio of 97.2%.

### <Synthesis Step of Fluorine-Containing Ether Compound>

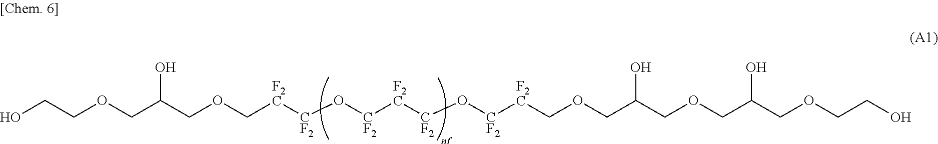

The compound (A¹) represented by the above formula (A¹) (nf in formula (A¹) which denoted an average degree of polymerization was 3.06) was obtained by the method shown below.

First, ethylene glycol monoallyl ether was reacted with 3,4-dihydro-2H-pyran to protect a hydroxy group with a tetrahydropyranyl group, and the double bond was then oxidized using metachloroperbenzoic acid to synthesize the compound represented by the following formula (A²). Also, the compound represented by the following formula (A⁴) was synthesized by the method shown below. The primary hydroxy group of 3-allyloxy-1,2-propanediol was protected with a t-butyldimethylsilyl group. Thereafter, the secondary hydroxy group was protected with a methoxymethyl group, and the t-butyldimethylsilyl group was removed from the obtained compound to synthesize the compound represented by the following formula (A³). The obtained compound (A³) was reacted with 2-(2-chloroethoxy)tetrahydropyran, and the double bond was then oxidized using m-chloroperbenzoic acid to synthesize the compound represented by the following formula (A⁴).

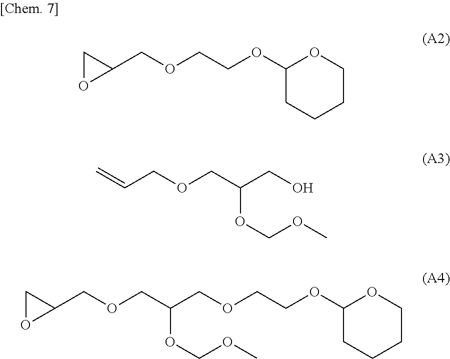 [SMILES: C1CCC(OCCOCC2CO2)OC1 ; C=CCOCC(CO)OCOC ; COCOC(COCCOC1CCCCO1)COCC1CO1]

A 200-mL eggplant flask was charged with the fluorinated 1, 3-propanediol pentamer HO—CH₂—CF₂CF₂O—(CF₂CF₂CF₂O)_{nd}—CF₂CF₂—CH₂—OH (nd represents an average degree of polymerization. nd=2.96 and Mn 769) (23.5 g), the compound represented by the above formula (A²) (3.71 g), and t-BuOH (tertiary butyl alcohol) (40.0 mL) under a nitrogen gas atmosphere, and the mixture was stirred at room temperature until it was homogenous. Furthermore, the eggplant flask was added with t-BuOK (potassium tertiary butoxide) (1.03 g), and the mixture was heated to 70° C. and stirred for 12 hours to carry out a reaction.

Thereafter, the obtained reaction product was cooled to 25° C., water was added, and ethyl acetate was further added as a solvent to extract an organic layer, which was then washed with water. Anhydrous sodium sulfate was added to the organic layer to dehydrate it, the drying agent was filtered off, and the filtrate was concentrated. The residue was purified by silica gel column chromatography to yield the compound represented by the following formula (A⁵) (11.9 g, yield 40%).

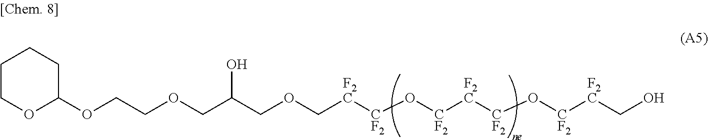

(wherein ne denoting an average degree of polymerization, is 3.06.)

A 200-mL eggplant flask was charged with the compound represented by formula (A⁵) (11.9 g), the compound represented by formula (A⁴) (4.31 g), and t-BuOH (tertiary butyl alcohol) (65.0 mL) under a nitrogen gas atmosphere, and the mixture was stirred at room temperature until it was homogeneous. Furthermore, the eggplant flask was added with t-BuOK (potassium tertiary butoxide) (0.41 g), and the mixture was heated to 70° C. and stirred for 16 hours to carry out a reaction.

Then, the reaction product obtained was cooled to 25° C., and a 7% hydrogen chloride/methanol reagent (63.7 g) was added, and the mixture was stirred at room temperature for 3 hours to carry out a deprotection reaction.

The reaction product obtained was neutralized by adding 7% sodium bicarbonate water (250 mL), and ethyl acetate was then added to extract an organic layer, which was washed with water. Anhydrous sodium sulfate was added to the organic layer to dehydrate it, the drying agent was filtered off, and the filtrate was concentrated. The residue was purified by silica gel column chromatography to yield the compound represented by the above formula (A¹) (9.24 g, yield 70%, nf representing an average degree of polymerization=3.06, and Mn 1,096).

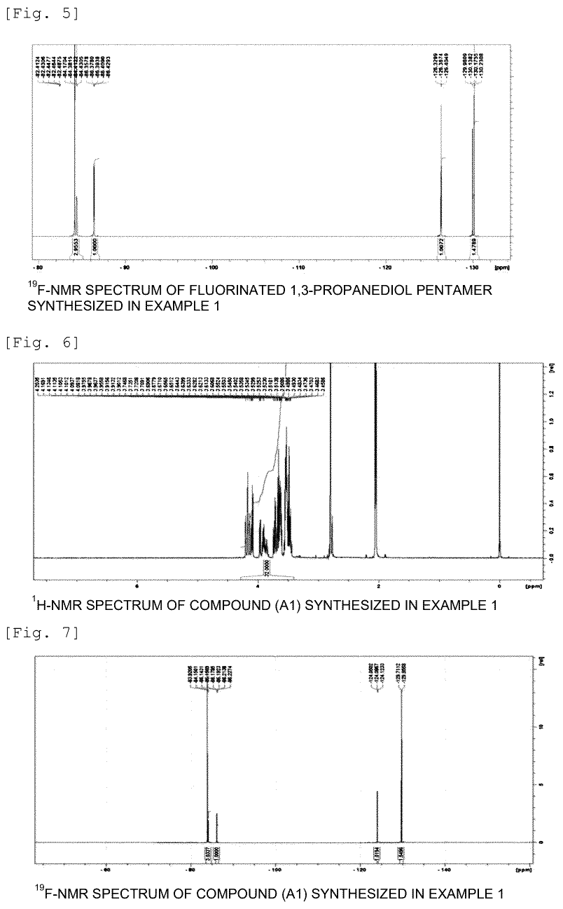

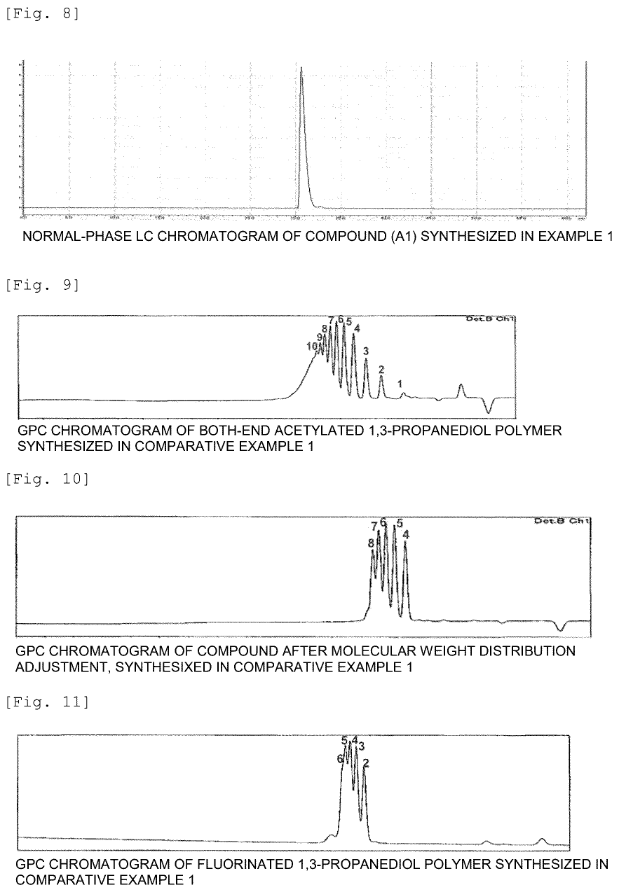

FIG. 6 and FIG. 7 show the ¹H-NMR and ¹⁹F-NMR spectra of the compound (A¹), respectively. A component with the degree of polymerization of 3 determined by the method under the HPLC conditions (ii) was a component having the largest peak area ratio, with an area ratio of 97.3%. FIG. 8 shows the normal-phase LC chromatogram measured by the method under the HPLC conditions (ii).

### Comparative Example 1

<Step of Introducing Protective Group to Hydroxy Groups at Both Ends>HO—(CH₂CH₂CH₂O)_{ng}—H (ng represents an average degree of polymerization) synthesized by step-growth polymerization was reacted with acetyl chloride to acetylate the hydroxy groups at both ends. The obtained CH₃—(C═O)—O—(CH₂CH₂CH₂O)_{ng}—(C═O)—CH₃ (ng represents an average degree of polymerization. ng=5.39) was used as a compound to be adjusted in the next molecular weight distribution adjustment step. A component with the degree of polymerization of 6 (CH₃—(C═O)—O—(CH₂CH₂CH₂O)₆—(C═O)—CH₃) determined by the method under the HPLC conditions (i) was a component having the largest peak area ratio, with an area ratio of 11.26%. FIG. 9 shows the GPC chromatogram measured by the method under the HPLC conditions (i). The numerical values in the graph represent assignments of degrees of polymerization of each peak. Note, however, peaks with a degree of polymerization of 11 or more could not be assigned.

### <Molecular Weight Distribution Adjustment Step>

100 g of the compound to be adjusted was fractionated in two times by preparative chromatography (500 g of silica, normal hexane/ethyl acetate=90/10 to 0/100 (volume ratio)). Components with degrees of polymerization from 4 to 8 were fractionated to obtain, as a raw material compound, a compound with an adjusted molecular weight distribution, CH₃—(C═O)—O—(CH₂CH₂CH₂O)ₙₕ—(C═O)—CH₃ (nh represents an average degree of polymerization. nh=5.66, Mn 431, and Mw/Mn=1.05) (rate of collection (mass ratio) 51%). A component with the degree of polymerization of 6 (CH₃—(C═O)—O—(CH₂CH₂CH₂O)₆—(C═O)—CH₃), determined by the method under the HPLC conditions (i), was a component with the largest peak area ratio, with an area ratio of 21.98%.

FIG. 10 shows the GPC chromatogram measured by the method under the HPLC conditions (i). The numerical values in the graph represent assignments of degrees of polymerization of each peak.

### <Liquid-Phase Fluorination Step (A)>

A 5L autoclave was introduced with 3,100 mL of HFTCB and then sealed. The autoclave was introduced with a nitrogen gas until internal pressure reached 0.3 MPa, and an operation of slowly releasing the internal pressure to normal pressure was repeated 10 times to purge the autoclave. 49 g of CH₃—(C═O)—O—(CH₂CH₂CH₂O)ₙₕ—(C═O)—CH₃ (nh represents an average degree of polymerization. nh=5.66, Mn 431, and Mw/Mn=1.05) obtained in the molecular weight distribution adjustment step was dissolved in 18.8 mL of HFTCB to obtain a raw material solution. A fluorine gas and a nitrogen gas were distributed at 588 mL/min and 4,600 mL/min, respectively, and while cooled to an internal temperature of 25° C., the raw material solution was introduced and distributed at a flow rate of 0.22 g/min on a solution mass basis, to carry out a fluorination reaction.

After the raw material solution was introduced and distributed, 1.87 g of C₆F₆ was dissolved in 73 mL of HFTCB to prepare a C₆F₆ solution. A temperature inside the autoclave was adjusted to 25 to 30° C., and while a fluorine gas was distributed at 150 mL/min and a nitrogen gas at 1,350 mL/min, the C₆F₆ solution was distributed and introduced at a flow rate of 0.83 g/min on a solution mass basis to carry out a final fluorination reaction.

<Step (B) of Reacting Fluorinated Reactant with Alcohol>

After completion of introduction of C₆F₆ in the step (A), the fluorine gas and nitrogen gas continued to be distributed for 10 minutes, and thereafter the fluorine gas stopped being distributed, and the nitrogen gas was distributed at 1,350 mL/min for 1 hour to purge an inside of the autoclave. While distributing the nitrogen gas, 73 g of methanol was introduced.

A reaction solution collected from the autoclave was washed with sodium carbonate water. After separation of the solution into two layers, an organic solvent layer was collected, dried with sodium sulfate and sodium fluoride, and then the solid was filtered off. The solvent was removed using an evaporator to obtain 106 g of product (rate of collection 99% (rate of collection relative to the raw material compounds of the fluorination reaction in the step (A))). As a result of having analyzed the product by ¹⁹F-NMR, it was confirmed to be CH₃O—(C═O)—CF₂CF₂O—(CF₂CF₂CF₂O)ₙᵢ—CF₂CF₂—(C═O)—OCH₃ (ni represents an average degree of polymerization. ni=3.82 and Mn 968).

<Step (C) of Obtaining Fluorinated Polyalkylene Glycol by Reduction Reaction>A reduction reaction of CH₃O—(C═O)—CF₂CF₂O—(CF₂CF₂CF₂O)ₙᵢ—CF₂CF₂—(C═O)—OCH₃ obtained in the step (B) was carried out using the following method.

A eggplant flask was introduced with 312 g of ethanol, cooled to 0° C., and charged with 5.28 g of sodium borohydride. While cooling the solution to 0° C., 104 g of CH₃O—(C═O)—CF₂CF₂O—(CF₂CF₂CF₂O)ₙᵢ—CF₂CF₂—(C═O)—OCH₃ (ni represents an average degree of polymerization. ni=3.82 and Mn 968) was added dropwise at 2.7 g/min, and the mixture was washed down with 35 g of ethanol. The reaction was allowed to continue for 4.5 hours while the eggplant flask was brought to room temperature.

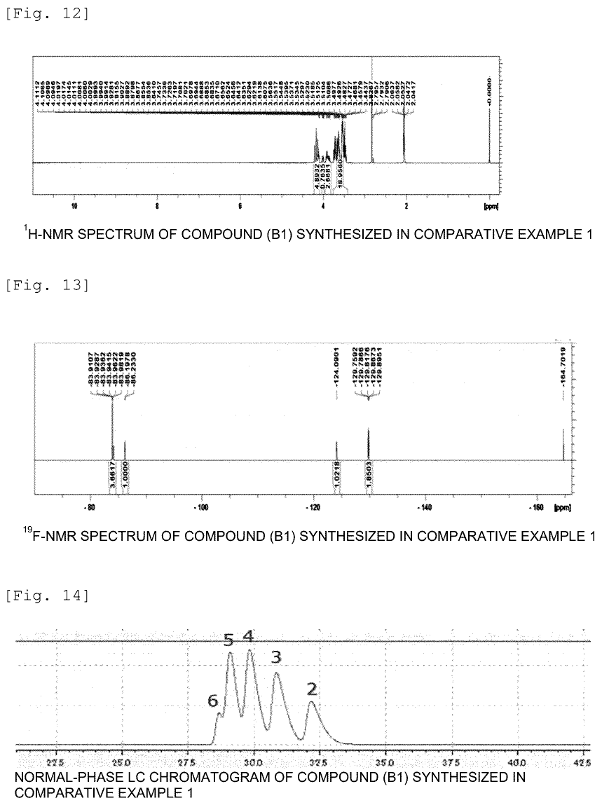

After completion of the reaction, 37 mL of 4M hydrochloric acid was added dropwise, and the pH of the solution was confirmed to be 3, and then a sodium hydrogen carbonate aqueous solution (19.1 g of sodium hydrogen carbonate and 220 mL of water) was added to confirm that the pH of the solution was 8. Ethanol was removed using an evaporator, 80 mL of water was added, and then an aqueous layer was extracted three times with 200 mL of AE-3000. Organic layers were combined and dried over magnesium sulfate, and the solids were then filtered off. The filtrate was concentrated under reduced pressure using an evaporator, yielding 96.6 g of crude product (rate of collection 98.4%). The crude product was purified by silica gel column chromatography, yielding 76.4 g (84.6 mmol, yield 78%) of a fluorinated 1,3-propanediol polymer HO—CH₂—CF₂CF₂O—(CF₂CF₂CF₂O)—CF₂CF₂—CH₂—OH (nj represents an average degree of polymerization. nj=3.77 and Mn 904) as colorless oil. A component (HO—CH₂—CF₂CF₂O—(CF₂CF₂CF₂O)₄—CF₂CF₂—CH₂—OH) with the degree of polymerization of 4, determined by the method under the HPLC conditions (i) was a component having the largest peak area ratio, with an area ratio of 22.6%. FIG. 11 shows the GPC chromatogram measured by the method under the HPLC conditions (i). The numerical values in the graph represent assignments of degrees of polymerization of each peak.

### <Synthesis Step of Fluorine-Containing Ether Compound>

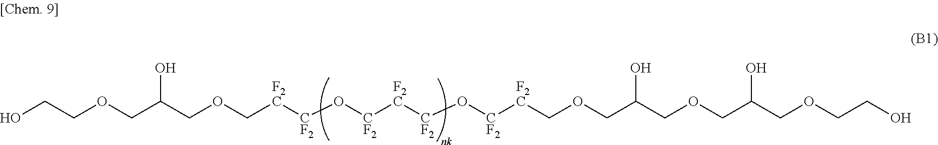

The compound (B1) represented by the above formula (B1) (wherein nk indicating an average degree of polymerization was 3.66) was obtained by the method described below.

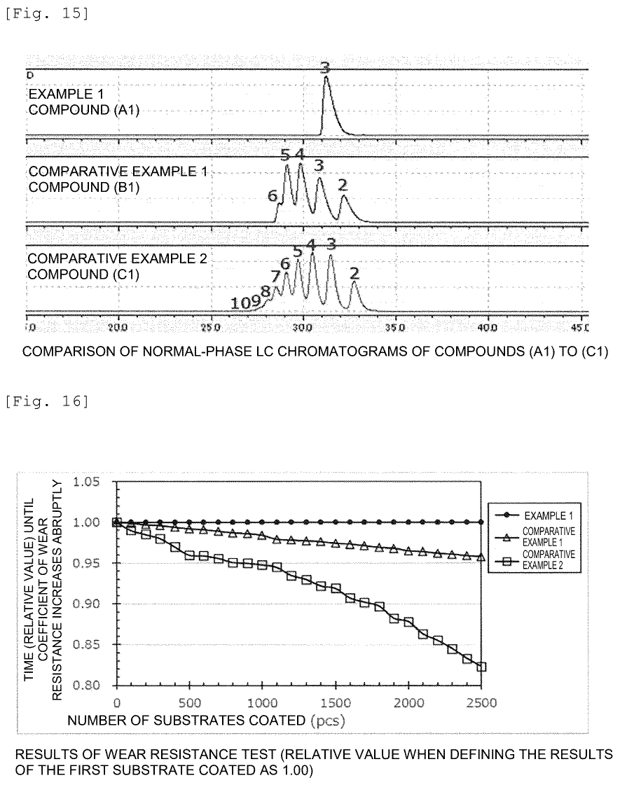

The same procedure as in Example 1 was carried out, except that the fluorinated 1,3-propanediol polymer HO—CH₂—CF₂CF₂O—(CF₂CF₂CF₂O)ₙⱼ—CF₂CF₂—CH₂—OH (nj represents an average degree of polymerization. nj=3.77 and Mn 904) was used instead of the fluorinated 1,3-propanediol pentamer HO—CH₂—CF₂CF₂O—(CF₂CF₂CF₂O)_{nd}—CF₂CF₂—CH₂—OH (nd represents an average degree of polymerization. nd=2.96 and Mn 769) in Example 1, to obtain the compound represented by the above formula (B1) (nk representing an average degree of polymerization=3.66 and Mn 1,196). The 1H-MJR and ¹⁹F-NMR spectra of compound (B1) are shown in FIG. 12 and FIG. 13, respectively. A component with the degree of polymerization of 4, determined by the method under the HPLC conditions (ii) was a component having the largest peak area ratio, with an area ratio of 29.8%. FIG. 14 shows the normal-phase LC chromatogram measured by the method under the HPLC conditions (ii). The numerical values in the graph represent assignments of degrees of polymerization of each peak.

### Comparative Example 2

HO—(CH₂CH₂CH₂O)ₙ₁—H (nl represents an average degree of polymerization) synthesized by the step-growth polymerization was reacted with acetyl chloride to acetylate the hydroxy groups at both ends. The reactions were carried out in the same manner as in the steps (A) to (C) of Comparative Example 1, except that 48 g of the resulting CH₃—(C═O)—O—(CH₂CH₂CH₂O)ₙₘ—(C═O)—CH₃ (nm represents an average degree of polymerization. nm=4.68, Mn 374, and Mw/Mn=1.44) was directly subjected to the step (A) without having adjusted the molecular weight distribution, to yield 36 g of product of step (C). As a result of having analyzed the product obtained by ¹⁹F-NMR, it was confirmed to be a fluorinated 1,3-propanediol polymer HO—CH₂—CF₂CF₂O—(CF₂CF₂CF₂O)ₙₙ—CF₂CF₂—CH₂—OH (nn represents an average degree of polymerization. nn=4.38 and Mn 1005).

### <Synthesis Step of Fluorine-Containing Ether Compound>

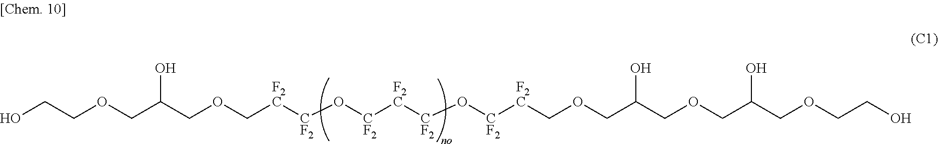

The compound (C1) represented by the above formula (C1) (wherein no, which indicates an average degree of polymerization was 4.35) was obtained by the method described below.

The same procedure as in Example 1 was carried out, except that the fluorinated 1,3-propanediol polymer HO—CH₂—CF₂CF₂O—(CF₂CF₂CF₂O)ₙₙ—CF₂CF₂—CH₂—OH (nn represents an average degree of polymerization. nn=4.38 and Mn 1,005) was used instead of the fluorinated 1,3-propanediol pentamer HO—CH₂—CF₂CF₂O—(CF₂CF₂CF₂O)_{nd}—CF₂CF₂—CH₂—OH (nd represents an average degree of polymerization. nd=2.96 and Mn 769) in Example 1, to obtain the compound represented by the above formula (C1) (no represents an average degree of polymerization=4.35 and Mn 1,310). A component with the degree of polymerization of 3, determined by the method under the HPLC conditions (ii) was a component having the largest peak area ratio, with an area ratio was 22.1%.

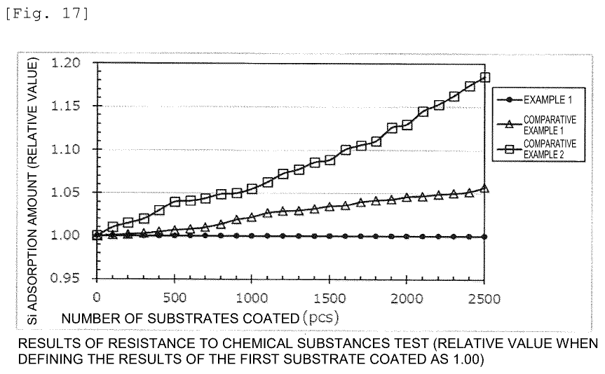

FIG. 15 shows a view comparing normal phase LC chromatograms of the compounds (A¹) to (C1) measured by the method under the HPLC conditions (ii). The numerical values in the graph represent assignments of degrees of polymerization of each peak.

Next, lubricating layer forming solutions were prepared using the compounds obtained in Example 1 and Comparative Examples 1 and 2 by the method described below. Then, using the obtained lubricating layer forming solutions, lubricating layers for magnetic recording media were formed by the method described below to obtain the magnetic recording media of Examples and Comparative Example. Thicknesses of the lubricating layers in the present Examples were measured by the method described below.

### <Thickness Measurement of Lubricating Layer>

Using a Fourier transform infrared spectrophotometer (FT-IR, product name: Nicolet iS50, manufactured by Thermo Fisher Scientific Inc.), a peak height of C—F stretching vibration of the lubricating layer was measured. Then, using a correlation equation obtained by the method described below, a thickness of the lubricating layer was calculated from the measured value of the peak height of C—F stretching vibration of the lubricating layer.

### <Method for Acquiring Correlation Equation>

A disk was prepared in which an adhesive layer, a soft magnetic layer, a first underlayer, a second underlayer, a magnetic layer, and a protective layer were sequentially provided on a substrate with a diameter of 65 mm. Lubricating layers were formed on the protective layers of these disks with a thickness of 6 to 10 Å (in 1 Å increments), respectively.

Thereafter, for each disk on which the lubricating layer was formed, an ellipsometer was used to measure an increase in film thickness from a disk surface on which no lubricating layer was formed, which was taken as a thickness of the lubricating layer. Also, for each disk on which the lubricating layer was formed, a peak height in C—F stretching vibration was measured by FT-IR.

Then, a correlation equation between the peak height obtained by FT-IR and the film thickness of the lubricating layer obtained by using an ellipsometer was given.

### <Lubricating Layer Forming Solution>

The compounds obtained in Examples and Comparative Examples were each dissolved in a fluorine-based solvent, Vertrel (registered trademark) XF (product name, manufactured by Chemours-Mitsui Fluoroproducts Co., Ltd.), and diluted with Vertrel XF so that a film thickness when a protective layer was coated with the obtained solution, was 8.0 Å, to obtain a lubricating layer forming solution with a compound concentration of 0.001% by mass to 0.005% by mass.

### <Magnetic Recording Medium>

On a substrate with a diameter of 65 mm, an adhesive layer, a soft magnetic layer, the first underlayer, the second underlayer, a magnetic layer, and a protective layer were sequentially provided. The protective layer was made of nitrogenated carbon.

A surface of the protective layer in the substrate on which each layer up to the protective layer had been formed, was coated with each of the lubricating layer forming solutions of Examples and Comparative Examples by a dipping method. Thereafter, a magnetic recording medium which was coated with the lubricating layer forming solution was placed in a thermostatic bath at 120° C. and heat-treated for 10 minutes. As a result, a lubricating layer was formed on the protective layer to obtain each of the magnetic recording media of Examples and Comparative Examples.

The lubricating layer forming solution poured in an immersion bath of a dip coater was used for continuous coating work, and heat treatment was performed in the same manner, to obtain 2,500 pieces of magnetic recording media for each Example or Comparative Example. The dipping method was performed under the conditions of a dip speed of 10 mm/sec and a dip time of 30 sec. During the continuous coating of the 2,500 substrates, the thickness of the lubricating layer was always controlled to be 8.0 Å by adjusting a speed at which the substrate was pulled up from the dip bath.

The magnetic recording media of Examples and Comparative Examples thus obtained were subjected to a wear resistance test and resistance to chemical substances test by the following method for the first coated sheet, the 100th coated sheet, the 200th coated sheet, and the rest at 100-sheet intervals. The results are shown in FIG. 16 and FIG. 17.

### <Wear Resistance Test>

Using a pin-on-disk type friction wear tester, an alumina ball with a diameter of 2 mm was slid on the lubricating layer of the magnetic recording medium at a load of 40 gf and a sliding rate of 0.25 m/sec, and a coefficient of friction of a surface of the lubricating layer was measured. Then, a sliding time until the coefficient of friction of the surface of the lubricating layer dramatically increased was measured. The sliding time until the coefficient of friction suddenly increased was measured four times for the lubricating layer of each magnetic recording medium, and an average value thereof was used. The evaluation results of wear resistance were compared using a relative value when the sliding time until the coefficient of friction of the first coated sheet suddenly increased was taken as 1.00.

The time until the coefficient of friction dramatically increases can be used as an index of the wear resistance of the lubricating layer for the following reasons. This is because use of magnetic recording medium progresses wear of the lubricating layer thereof, and when the lubricating layer is worn away, a contact and the protective layer come into direct contact, resulting in a sharp increase in the coefficient of friction.

### <Resistance to Chemical Substances Test>

The following evaluation method was used to investigate contamination of magnetic recording media by environmental substances that create pollutants in high-temperature environments. In the following evaluation method, Si ions were used as the environmental substances, and the amount of Si adsorbed was measured as the amount of pollutants created by the environmental substances that contaminate the magnetic recording medium.

Specifically, a magnetic recording medium to be evaluated was held in the presence of siloxane-based Si rubber for 240 hours in a high-temperature environment of 85° C. and 0% humidity. The amount of Si adsorbed present on a surface of the magnetic recording medium was then analyzed and measured using secondary ion mass spectrometry (SIMS), and a degree of contamination by Si ions was evaluated as the amount of Si adsorbed. The evaluation results of the amount of Si adsorbed were compared using a relative value when the amount of Si adsorbed on the first substrate coated was taken as 1.00.

As shown in FIG. 16 and FIG. 17, it was confirmed that the magnetic recording medium of Example 1 could maintain the wear resistance and resistance to chemical substances of the lubricating layer without deteriorating in spite of the increased number of substrates coated. On the other hand, Comparative Example 1 and Comparative Example 2 could confirm that the wear resistance and resistance to chemical substances deteriorated as the number of substrates coated increased. Comparing Comparative Example 1 and Comparative Example 2, it was also confirmed that Comparative Example 1, which included the molecular weight distribution adjustment step, was able to significantly inhibit the wear resistance and resistance to chemical substances from deteriorating, compared to Comparative Example 2 in which the molecular weight distribution was not adjusted.

This can be interpreted as the fact that when using the conventional fluorine-containing ether compound having a wide molecular weight distribution as the lubricant as in Comparative Example 2, the number-average molecular weight of the fluorine-containing ether compound contained in the lubricant layer increased as the number of substrates coated increased, resulting in the sudden deterioration in the wear resistance and resistance to chemical substances.

When using the fluorine-containing ether compound having no molecular weight distribution and having a proportion of molecules with the same molecular weight of 90% or more as the lubricant as in Example 1, the number-average molecular weight of the fluorine-containing ether compound contained in the lubricating layer hardly changes, thereby enabling maintaining the wear resistance and resistance to chemical substances of the lubricating layer without deteriorating.

### REFERENCE SIGNS LIST

10. magnetic recording medium
11. substrate
12. adhesive layer
13. soft magnetic layer
14. first underlayer
15. second underlayer
16. magnetic layer
17. protective layer
18. lubricating layer

1. A lubricant for magnetic recording media, comprising a fluorine-containing ether compound represented by formula (1), wherein a proportion of molecules with the same molecular weight contained in the fluorine-containing ether compound is 90% or more in terms of a peak area ratio based on high-performance liquid chromatography: 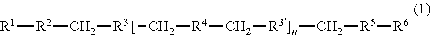
wherein n is any one of integers of 0 to 2; R³ and R^{3′} are each independently a perfluoropolyether chain represented by formula (1f); when n is 1 or 2, R³ and one or two R^{3′}s are partially or totally the same, or are different from each other; R⁴ is a divalent linking group having 1 to 3 polar groups; when n is 2, two R⁴s are the same or different; R² and R⁵ are divalent linking groups having one or more polar groups, and are the same or different; an end of R² on a side bonded to R¹ is an oxygen atom; an end of R⁵ on a side bonded to R⁶ is an oxygen atom; R¹ and R⁶ are each a terminal group bonded to an oxygen atom at an end of R² or R⁵, and are the same or different; and R¹ and R⁶ are hydrogen atoms or organic groups having 1 to 50 carbon atoms, 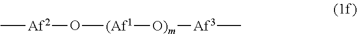
wherein Af¹ represents a divalent perfluorohydrocarbon group having a linear or branched structure having 2 to 5 carbon atoms; when Af¹ has a linear structure, repeating units represented by (Af¹—O) are all the same; when Af¹ has a branched structure, a structure in which an orientation direction of Af¹ in the repeating unit represented by (Af¹—O) is interchanged from left to right is optionally partially included; Af² and Af³ each independently represent a divalent linear perfluorohydrocarbon group having 1 to 4 carbon atoms; and m represents an average degree of polymerization and is a real number of 1 to 13.

2. A lubricant for magnetic recording media, comprising a fluorine-containing ether compound represented by formula (1), wherein a proportion of molecules represented by formula (1s) to 100% of the fluorine-containing ether compound represented by formula (1) is 90% or more in terms of a peak area ratio based on high-performance liquid chromatography: 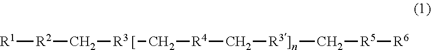
wherein n is any one of integers of 0 to 2; R³ and R^{3′} are each independently a perfluoropolyether chain represented by formula (1f); when n is 1 or 2, R³ and one or two R^{3′}s are partially or totally the same, or are different from each other; R⁴ is a divalent linking group having 1 to 3 polar groups; when n is 2, two R⁴s are the same or different; R² and R⁵ are divalent linking groups having one or more polar groups, and are the same or different; an end of R² on a side bonded to R¹ is an oxygen atom; an end of R⁵ on a side bonded to R⁶ is an oxygen atom; R¹ and R⁶ are each a terminal group bonded to an oxygen atom at an end of R² or R⁵, and are the same or different; and R¹ and R⁶ are hydrogen atoms or organic groups having 1 to 50 carbon atoms, 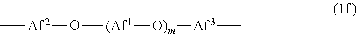
wherein Af¹ represents a divalent perfluorohydrocarbon group having a linear or branched structure having 2 to 5 carbon atoms; when Af¹ has a linear structure, repeating units represented by (Af¹—O) are all the same; when Af¹ has a branched structure, a structure in which an orientation direction of Af¹ in the repeating unit represented by (Af¹—O) is interchanged from left to right is optionally partially included; Af² and Af³ each independently represent a divalent linear perfluorohydrocarbon group having 1 to 4 carbon atoms; and m represents an average degree of polymerization and is a real number of 1 to 13, 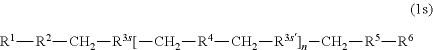
wherein the same symbols as in formula (1) represent the same structures or values as in formula (1); and R^{3s} and R^{3s′} are each independently a perfluoropolyether chain represented by formula (1f-1): 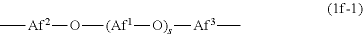
wherein the same symbols as in formula (1f) represent the same structures as in formula (1f); and s represents a degree of polymerization and is any one of integers of 1 to 13.

3. The lubricant for magnetic recording media according to claim 1, wherein a number-average molecular weight of the fluorine-containing ether compound is 500 to 10,000.

4. A method for producing a lubricant for magnetic recording media, the lubricant comprising a fluorine-containing ether compound represented by formula (1), wherein a proportion of molecules with the same molecular weight contained in the fluorine-containing ether compound is 90% or more in terms of a peak area ratio based on high-performance liquid chromatography,
the method comprising a step of synthesizing the fluorine-containing ether compound using a fluorinated polyalkylene glycol represented by formula (1f′) as a raw material;
wherein a proportion of molecules with the same molecular weight contained in the fluorinated polyalkylene glycol is 90% or more in terms of a peak area ratio based on high-performance liquid chromatography: 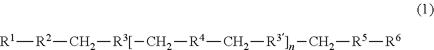
wherein n is any one of integers of 0 to 2; R³ and R^{3′} are each independently a perfluoropolyether chain represented by formula (1f); when n is 1 or 2, R³ and one or two R^{3′}s are partially or totally the same, or are different from each other; R⁴ is a divalent linking group having 1 to 3 polar groups; when n is 2, two R⁴s are the same or different; R² and R⁵ are divalent linking groups having one or more polar groups, and are the same or different; an end of R² on a side bonded to R¹ is an oxygen atom; an end of R⁵ on a side bonded to R⁶ is an oxygen atom; R¹ and R⁶ are each a terminal group bonded to an oxygen atom at an end of R² or R⁵, and are the same or different; and R¹ and R⁶ are hydrogen atoms or organic groups having 1 to 50 carbon atoms, 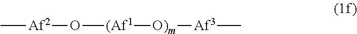
wherein Af¹ represents a divalent perfluorohydrocarbon group having a linear or branched structure having 2 to 5 carbon atoms; when Af¹ has a linear structure, repeating units represented by (Af¹—O) are all the same; when Af¹ has a branched structure, a structure in which an orientation direction of A^{f} in the repeating unit represented by (Af¹—O) is interchanged from left to right is optionally partially included; Af² and Af³ each independently represent a divalent linear perfluorohydrocarbon group having 1 to 4 carbon atoms; and m represents an average degree of polymerization and is a real number of 1 to 13, 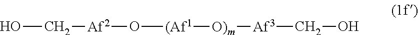
wherein Af¹, Af², Af³ and m correspond to those in formula (1f); and in formula (1), when n is 1 or 2 and when R³ and one or two R^{3′}s are not all the same, formula (1f′) represents a fluorine-containing polyalkylene glycol corresponding to each of R³ and R^{3′}.

5. The method for producing a lubricant for magnetic recording media according to claim 4, wherein the fluorinated polyalkylene glycol represented by formula (1f) is synthesized by a method comprising the following steps (A) to (C):
step (A) of liquid-phase fluorinating a both-end-protected polyalkylene glycol represented by formula (2) in which a proportion of molecules with the same molecular weight is 90% or more in terms of a peak area ratio based on high-performance liquid chromatography,
step (B) of reacting the obtained fluorinated reactant represented by formula (3) with an alcohol having 1 to 6 carbon atoms, and
step (C) of carrying out a reduction reaction of the obtained both-end esterified product represented by formula (4), 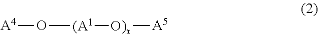
wherein A¹ represents a divalent hydrocarbon group having a linear or branched structure having 2 to 5 carbon atoms; when A¹ has a linear structure, repeating units represented by (A¹—O) are all the same; when A¹ has a branched structure, a structure in which an orientation direction of A¹ in the repeating unit represented by (A¹—O) is interchanged from left to right is optionally partially included; x represents an average degree of polymerization and is a real number of 3 to 15; and A⁴ and A⁵ each independently represent a protecting group for a hydroxy group, 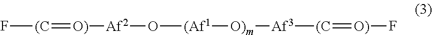
wherein Af¹, Af², Af³ and m correspond to those in formula (1f), 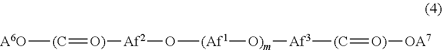
wherein Af¹, Af², Af³ and m correspond to those in formula (1f); and A⁶ and A⁷ each independently represent an alkyl group having 1 to 6 carbon atoms.

6. The method for producing a lubricant for magnetic recording media according to claim 5, comprising, prior to the step (A), a polyether synthesis step of carrying out a nucleophilic substitution reaction of reacting two or more compounds having a polyether chain or a monomer unit constituting the polyether chain to synthesize a polyether compound having a repeating unit represented by (A¹—O).

7. A method for producing a lubricant for magnetic recording media, the lubricant comprising a fluorine-containing ether compound represented by formula (1), wherein a proportion of molecules represented by formula (1s) to 100% of the fluorine-containing ether compound represented by formula (1) is 90% or more in terms of a peak area ratio based on high-performance liquid chromatography,
the method comprising a step of synthesizing the fluorine-containing ether compound using a fluorinated polyalkylene glycol represented by formula (1f′) as a raw material,
wherein a proportion of molecules represented by formula (1f′-1) to 100% of the fluorinated polyalkylene glycol represented by formula (1f′) is 90% or more in terms of a peak area ratio based on high-performance liquid chromatography: 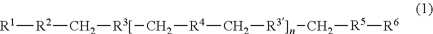
wherein n is any one of integers of 0 to 2; R³ and R^{3′} are each independently a perfluoropolyether chain represented by formula (1f); when n is 1 or 2, R³ and one or two R^{3′}s are partially or totally the same, or are different from each other; R⁴ is a divalent linking group having 1 to 3 polar groups; when n is 2, two R⁴s are the same or different; R² and R⁵ are divalent linking groups having one or more polar groups, and are the same or different; an end of R² on a side bonded to R¹ is an oxygen atom; an end of R⁵ on a side bonded to R⁶ is an oxygen atom; R¹ and R⁶ are each a terminal group bonded to an oxygen atom at an end of R² or R⁵, and are the same or different; and R¹ and R⁶ are hydrogen atoms or organic groups having 1 to 50 carbon atoms, 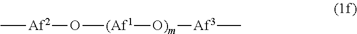
wherein Af¹ represents a divalent perfluorohydrocarbon group having 2 to 5 carbon atoms and having a linear or branched structure; when Af¹ has a linear structure, repeating units represented by (Af¹—O) are all the same; when Af¹ has a branched structure, a structure in which an orientation direction of A^{f} in the repeating unit represented by (Af¹—O) is interchanged from left to right is optionally partially included; Af² and Af³ each independently represent a divalent linear perfluorohydrocarbon group having 1 to 4 carbon atoms; and m represents an average degree of polymerization and is a real number of 1 to 13, 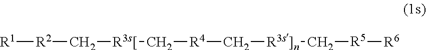
wherein the same symbols as in formula (1) represent the same structures or values as in formula (1); and R^{3s} and R^{3s′} are each independently a perfluoropolyether chain represented by formula (1f-1), 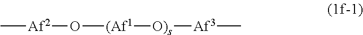
wherein the same symbols as in formula (1f) represent the same structures as in formula (1f); and s represents a degree of polymerization and is any one of integers of 1 to 13, 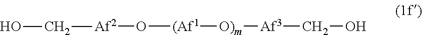
wherein Af¹, Af², Af³ and m correspond to those in formula (1f); and in formula (1), when n is 1 or 2 and when R³ and one or two R^{3′} are not all the same, formula (1f′) represents a fluorine-containing polyalkylene glycol corresponding to each of R³ and R^{3′}, 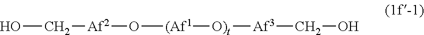
wherein the same symbols as in formula (1f′) represent the same structures as in formula (1f′); and t represents a degree of polymerization and is any one of integers of 1 to 13.

8. The method for producing a lubricant for magnetic recording media according to claim 7, wherein the fluorinated polyalkylene glycol represented by formula (1f) is synthesized by a method comprising the following steps (A-1) to (C-1):
step (A-1) of liquid-phase fluorinating a both-end-protected polyalkylene glycol represented by formula (2), wherein a proportion of molecules represented by formula (2-1) is 90% or more in terms of a peak area ratio based on high-performance liquid chromatography;
step (B-1) of reacting the obtained fluorinated reactant represented by formula (3) with an alcohol having 1 to 6 carbon atoms; and
step (C-1) of carrying out a reduction reaction of the obtained both-end esterified product represented by formula (4), 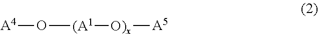
wherein A¹ represents a divalent hydrocarbon group having a linear or branched structure having 2 to 5 carbon atoms; when A¹ has a linear structure, repeating units represented by (A¹-O) are all the same; when A¹ has a branched structure, a structure in which an orientation direction of A¹ in the repeating unit represented by (A¹-O) is interchanged from left to right is optionally partially included; x represents an average degree of polymerization and is a real number of 3 to 15; and A⁴ and A⁵ each independently represent a protecting group for a hydroxy group, 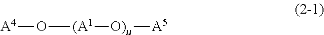
wherein the same symbols as in formula (2) represent the same structures as in formula (2); and u represents a degree of polymerization and is any one of integers of 3 to 15, 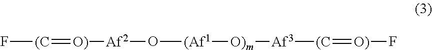
wherein Af¹, Af², Af³ and m correspond to those in formula (1f), 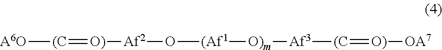
wherein Af¹, Af², Af³ and m correspond to those in formula (1f); and A⁶ and A⁷ each independently represent an alkyl group having 1 to 6 carbon atoms.

9. The method for producing a lubricant for magnetic recording media according to claim 8, comprising, prior to the step (A-1), a polyether synthesis step of carrying out a nucleophilic substitution reaction of reacting two or more compounds having a polyether chain or a monomer unit constituting the polyether chain to synthesize a polyether compound having a repeating unit represented by (A¹—O).

10. The method for producing a lubricant for magnetic recording media according to claim 6,
wherein the polyether synthesis step comprises a step of reacting a compound having a leaving group at one end of a polyether chain or a monomer unit constituting the polyether chain and a protected hydroxy group at the other end
with a compound having a hydroxy group at one end of a polyether chain or a monomer unit constituting the polyether chain and a protected hydroxy group at the other end.

11. The method for producing a lubricant for magnetic recording media according to claim 6,
wherein the polyether synthesis step comprises a step of reacting a compound having a leaving group at one end of a polyether chain or a monomer unit constituting the polyether chain and a protected hydroxy group at the other end
with a compound having hydroxy groups at both ends of a polyether chain or a monomer unit constituting the polyether chain.

12. The method for producing a lubricant for magnetic recording media according to claim 6,
wherein the polyether synthesis step comprises a step of reacting a compound having a hydroxy group at one end of a polyether chain or a monomer unit constituting the polyether chain and a protected hydroxy group at the other end
with a compound having leaving groups at both ends of a polyether chain or a monomer unit constituting the polyether chain.

13. A magnetic recording medium, comprising at least a magnetic layer, a protective layer, and a lubricating layer sequentially provided on a substrate, wherein the lubricating layer comprises the lubricant for magnetic recording media according to claim 1.

14. A method for producing a magnetic recording medium, comprising a step of coating a surface of a protective layer in a substrate on which at least a magnetic layer and the protective layer are formed, with the lubricant for magnetic recording media according to claim 1 to form a lubricating layer.
